(19) United States
(12) Patent Application Publication
(10) Pub. No.: US 2006/0160114 A1
(43) Pub. Date: Jul. 20, 2006

(54) Reagents and methods for predicting drug resistance

(75) Inventors: Christopher Kerfoot, Mission Viejo, CA (US); William A. Ricketts, Irvine, CA (US); David L. Smith, Santa Ana, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Oncotech, Inc., Tustin, CA (US)

(21) Appl. No.: 11/293,529

(22) Filed: Dec. 2, 2005

Related U.S. Application Data
(60) Provisional application No. 60/632,617, filed on Dec. 2, 2004.

(51) Int. Cl. C12Q 1/68; G01N 33/537; C12P 19/34

(52) U.S. Cl. 435/6; 435/91.2; 435/7.92

(57) ABSTRACT

The invention provides methods for prognosis, diagnosis, staging and determining disease progression in human cancer patients related to expression levels of one or a plurality of genes or genetic loci that are differentially deleted, amplified, expressed or amplified and over-expressed in chemotherapeutic drug resistant tumor cells.

This application claims priority to U.S. provisional application Ser. No. 60/632,617, filed Dec. 2, 2004.

### BACKGROUND OF THE INVENTION

1. Field of the Invention

The invention relates to cancer diagnosis and treatment, and specifically to the determination of a drug resistance phenotype in neoplastic cells from cancer patients. The invention in particular relates to the identification of regions within tumor cell DNA that are amplified or deleted in tumors that are resistant to specific chemotherapeutic agents, specifically breast cancer, ovarian cancer, and non-small cell lung cancer cells. The invention provides a plurality of genes and genetic loci that are genetically amplified and/or exhibit an increase in drug resistant neoplastic cells. In addition, the invention provides a plurality genes and genetic loci that are deleted in drug-resistant neoplastic cells. The invention provides methods for identifying such genes or expression patterns of such genes that are increased in drug resistant phenotypes and methods for identifying such genetic loci that are amplified or deleted as well as methods for using this information to make clinical decisions on cancer treatment, especially chemotherapeutic drug treatment of cancer patients.

2. Summary of the Related Art

Cancer remains one of the leading causes of death in the United States. Clinically, a broad variety of medical approaches, including surgery, radiation therapy and chemotherapeutic drug therapy are currently being used in the treatment of human cancer (see the textbook CANCER: Principles & Practice of Oncology, 2d Edition, De Vita et al., eds., J.B. Lippincott Company, Philadelphia, Pa., 1985). However, it is recognized that such approaches continue to be limited by a fundamental inability to accurately predict the likelihood of clinically successful outcomes, particularly with regard to the sensitivity or resistance of a particular patient's tumor to a chemotherapeutic agent or combinations of chemotherapeutic agents.

A broad variety of chemotherapeutic agents are used in the treatment of human cancer. These include the plant alkaloids vincristine, vinblastine, vindesine, and VM-26; the antibiotics actinomycin-D, doxorubicin, daunorubicin, mithramycin, mitomycin C and bleomycin; the antimetabolites methotrexate, 5-fluorouracil, 5-fluorodeoxyuridine, 6-mercaptopurine, 6-thioguanine, cytosine arabinoside, 5-aza-cytidine and hydroxyurea; the alkylating agents cyclophosphamide, melphalan, busulfan, CCNU, MeCCNU, BCNU, streptozotocin, chlorambucil, bis-diamminedichloroplatinum, azetidinylbenzoquinone; and the miscellaneous agents dacarbazine, mAMSA and mitoxantrone (De Vita et al., Id.). However, some neoplastic cells become resistant to specific chemotherapeutic agents, in some instances even to multiple chemotherapeutic agents, and some tumors are intrinsically resistant to certain chemotherapeutic agents. Such drug resistance or multiple drug resistance can theoretically arise from expression of genes that confer resistance to the agent, or from lack of expression of genes that make the cells sensitive to a particular anticancer drug. One example of the former type is the multidrug resistance gene, MDR1, which encodes an integral plasma membrane protein termed P-glycoprotein that is a non-specific, energy-dependent efflux pump. (See Roninson (ed)., 1991, Molecular and Cellular Biology of Multidrug Resistance in Tumor Cells, Plenum Press, N.Y., 1991; Gottesman et al., 1991, in Biochemical Bases for Multidrug Resistance in Cancer, Academic Press, N.Y., Chapter 11 for reviews). Examples of the latter type include topoisomerase II, the expression of which makes cells sensitive to the anticancer drug etoposide. Decreased expression of this enzyme makes neoplastic cells resistant to this drug. (See Gudkov et al., 1993, Proc. Natl. Acad. Sci. USA 90: 3231-3235). Although these are just single examples of the way that modulation of gene expression can influence chemotherapeutic drug sensitivity or resistance in neoplastic cells, these examples demonstrate the diagnostic and prognostic potential for identifying genes the expression of which (or the pattern of gene expression modulation thereof) are involved in mediating the clinical effectiveness of anticancer drug treatment.

Drug discovery programs have evolved to include rational therapeutic development strategies in addition to traditional empirical screening approaches. Rational therapy development focuses on the identification of specific pathways that are differentially activated in cancer cells compared to normal tissue (Bichsel et al., 2001, Cancer J. 7: 69-78; Winters, 2000, Curr. Opin. Mol. Ther. 2: 670-681). Such selective targeting can significantly reduce therapy-associated toxicity. Recent examples where this approach has led to the successful development of new anti-cancer agents include targeting HER2 with Herceptin (Bange et al., 2001, Nat. Med. 7: 548-552) in breast cancer and Gleevec's (ST1571) inhibition of the BCR-abl kinase fusion protein in chronic myeloid leukemia (2001, Oncology (Huntingt) 15: 23-31).

Unfortunately, cancer specific pathways are not universal to the transformation process, which involves a variety of alterations in tumor suppressor genes, oncogenes, translocations, deletions and mutations. The genomic instability inherent to this pleiotropic background of metabolic alterations results in significant phenotypic heterogeneity within each tumor (Bertram, 2000, Mol. Aspects Med. 21: 167-223; Yamasaki et al., 2000, Toxicol. Lett. 112-113: 251-256). Treatment targets are therefore unstable, leading to intrinsic and acquired resistance to rationally designed agents.

One class of genetic alterations that occur in neoplastic transformation is DNA amplification. Amplifications of genes or genetic loci are a common event in breast cancer that has useful clinical implications. For example, amplification of the HER2 gene on chromosome 17q11.2-12 is predictive of response to Herceptin therapy, and fluorescence in situ hybridization (FISH) detection kits are commercially available. Amplification correlates reasonably well with increased HER2 transcript levels. Gene amplification may be related to drug sensitivity or resistance to chemotherapeutic agents. Amplification of Topoisomerase II-alpha (TOP2A) on chromosome 17q21-22 is also clinically relevant. TOPIIA is a key enzyme in DNA replication, cell cycle progression and chromosome segregation, and is correlated with resistance to anthracyclines.

Amplification of genetic loci in breast cancer has been studied by comparative genomic hybridization (CGH), and the frequency of chromosomal amplifications has been defined. The relationship between such CGH data and cancer chemotherapy is in its infancy, however, and markers similar to HER2 and TOPIIA have not yet been identified for other important breast cancer treatment drugs such as paclitaxel (Taxol) or docetaxel (Taxotere).

Thus, there is a need in this art for developing methods for identifying the amplification of genes or genetic loci that are predictive of the clinical effectiveness of anticancer drug treatment therapies, in order to make more informed decisions for treating individual cancer patients with anticancer drugs having greatest likelihood of producing a positive outcome.

In contrast to the relative recency of genetic approaches to identifying gene amplification and gene expression patterns that correlate with drug resistance or sensitivity, the art recognizes functional approaches to determining whether a specific cancer will respond to chemotherapeutic treatment. One example is the Extreme Drug Resistance (EDR®) assay. The EDR® Assay is an in vitro test that measures the ability of pharmaceutical agents and other chemotherapies to stop cancer cells from dividing and growing. This assay identifies patients that will not respond to a particular cancer therapeutic with >99% accuracy, and has been used in the art to exclude agents unlikely to be clinically effective from treatment of individual cancer patients, consequently sparing them the morbidity of ineffective chemotherapy. The EDR® Assay has also been used to select chemotherapeutic agents that have the greatest likelihood of being clinically effective, resulting in improved response rates and prolonged survival of cancer patients

Kern and Weisenthal (1990, J. Nat. Cancer Inst. 82: 582-588) showed that the EDR® Assay identified patients that would not respond to a cancer therapeutic. In this study, patients whose cancer cells demonstrated Extreme Drug Resistance to an anti-cancer drug had a greater than 99% failure rate when treated with that drug. In contrast, patients with Low Drug Resistance to an anti-cancer drug had a 52% clinical response rate when treated with that drug, which was significantly higher than the overall response rate to anti-cancer drugs of 29% (Id.).

In the practice of this assay, tumor cells are taken from a cancer biopsy and exposed to cancer chemotherapeutics in culture. During the culture period, radioactive thymidine is added, which is incorporated into the DNA of growing and dividing cancer cells (which are thus resistant to the cytostatic or cytotoxic effects of the cancer chemotherapeutic). Tritiated thymidine is not incorporated into cells that are sensitive to the drug and reduce or suspend growth and division in response to the drug. Since cells affected by anticancer drugs do not divide, or divide more slowly, they therefore incorporate lesser amounts of the radioactive thymidine. By measuring the amount of radioactivity in a sample, the assay determines the relative resistance of an individual patient's cancer cells to a number of different chemotherapies.

The EDR® Assay is highly accurate at predicting clinically inactive drugs. Patients whose cancer cells have Extreme Drug Resistance to an anticancer agent have <1% response rate to that agent (Kern & Weisenthal, Id.). Clinical data suggest a therapeutic advantage in the activity of agents to which a tumor is highly active in vitro (Alberts et al., 1991, Anticancer Drugs 2:69-77), and treatment based on drugs that are active in vitro may improve response rates and survival (Kern & Weisenthal, Id.; Alberts et al., 1991, Anticancer Drugs 2:69-77; Kern, 1997, Cancer 79: 1447-50; Holloway et al., 2002, Gynecol Oncol 87: 8-16.; Mehta et al., 2001, Breast Cancer Res. Treat 66: 225-37).

Thus, there is a need in this art to determine the relationship between functional drug resistance or sensitivity as evaluated, inter alia, by cell assays such as the EDR® Assay, and changes in gene expression or DNA copy number that correlate with the clinical resistance to a given therapeutic agent.

### SUMMARY OF THE INVENTION

This invention provides methods and reagents for identifying changes in DNA copy number comprising one or a plurality of genes deleted, amplified and/or overexpressed in tumor samples, most preferably human tumor samples, wherein the genes or genetic loci are differentially deleted, amplified and/or overexpressed in drug resistant versus drug sensitive tumor samples. The invention also provides methods for determining a prognosis for an individual having a tumor, wherein the prognosis is particularly directed towards determining the likelihood that a particular treatment modality would be effective in treating the individual's cancer. The treatment modality is preferably a chemotherapeutic treatment, most preferably treatment with the anticancer drug paclitaxel or docetaxel. In certain embodiments the tumor is preferably a breast cancer tumor, ovarian cancer tumor, or non-small cell lung cancer tumor. The invention also provides one or a plurality of said deleted, amplified and/or overexpressed genes and one or a plurality of genetic loci that are altered for use in the prognostic methods of the invention.

Specific preferred embodiments of the present invention will become evident from the following more detailed description of certain preferred embodiments and the claims.

### BRIEF DESCRIPTION OF THE DRAWINGS

FIG. 1 is a histogram of the percent of breast cancer patients having tumors with amplified DNA correlated with the chromosome arm on which the amplified DNA is located. Note the high percentage of patients with amplified DNA on chromosome 17q, as expected in view of the frequency of HER2 gene amplification on that chromosome in breast cancer patients. An even higher percentage of breast cancer patients are shown in the histogram as having amplified DNA on the q arm of chromosome 1, consistent with the identification herein of five genes at that chromosomal location that are amplified and overexpressed in the majority of breast cancer patients studied. This histogram was prepared by comparative genomic hybridization as disclosed in Pollack et al. (2002, Proc. Natl. Acad Sci. USA 99: 12963-12968), and is limited to identifying genomic regions amplified in particular tumor types without further analysis of the genes contained in the amplified regions or gene expression levels of such genes.

FIG. 2 is a histogram of responders and non-responders among 450 tumor samples subjected to EDR® assay.

FIG. 3 is a graph showing survival of cancer patients bearing tumors showing extreme drug resistance (EDR) compared with patients having tumors showing intermediate or low drug resistance (I/LDR) in EDR® assay.

FIG. 4 is a histogram of normalized gene expression levels of five genes (H2BFQ, SLC19A2, ZNF281, DAF and ATF3) shown herein to be amplified and/or overexpressed in the majority of breast cancer tumor samples studies, for tumors determined to exhibit low drug resistance (LDR) and extreme drug resistance (EDR) in EDR® assay.

FIG. 5 is a pictogram representing a portion of chromosome 1q32 comprising the genes from DAF to ATF3. The region between DAF and ATF3 contains at least 32 genes and expressed sequence tags (ESTs) that may cause or contribute to paclitaxel resistance. This is an example of the number of genes or expressed sequence tags that are found in the regions between the genes described in Table 1.

FIG. 6 demonstrates the differences in gene expression as detected by polymerase chain reaction (PCR) in two different Taxol Low Drug Resistance (LDR) tumors and two Taxol Extreme Drug Resistant (EDR®) tumors. PCR primers were made to IL6R, H2BFQ, DAF, and ATF3 and PCR was performed on equal amounts of DNA. The results show that the genes are present in a higher amount in the Taxol EDR specimens.

FIGS. 7A and 7B are schematic diagrams of the protocol for the Comparative Genomic Hybridization arrays. The DNA is labeled with both Cy5 and Cy3 dyes.

FIG. 8 is an example of the results of CGH assay for chromosome 1 for two tumor samples. These diagrams demonstrate the signal from the CGH arrays plotted by physical location on the chromosome. These two tumors lost the 1p arm and gained the 1q arm.

FIG. 9 is an example of the Y chromosome control. Since these are ovarian tumors, no Y chromosome is expected or detected.

FIG. 10 is a graph indicating the number of tumors that gained, lost, or had no change within the regions of chromosome 1 listed. In addition, the data is separated by Taxol EDR and Taxol LDR tumors. These data show that gains on chromosome 1 become more selective for Taxol EDR tumors the further from the centromere that the probe is positioned on the q arm of the chromosome.

### DETAILED DESCRIPTION OF THE PREFERRED EMBODIMENTS

The present invention provides methods for making a prognosis about disease course in a human cancer patient. For the purposes of this invention, the term “prognosis” is intended to encompass predictions and likelihood analysis of disease progression, particularly tumor recurrence, metastatic spread and disease relapse. The prognostic methods of the invention are intended to be used clinically in making decisions concerning treatment modalities, including therapeutic intervention, diagnostic criteria such as disease staging, and disease monitoring and surveillance for metastasis or recurrence of neoplastic disease.

The invention also provides methods for identifying genes or genetic loci useful for making a cancer prognosis, by virtue of said genes being differentially deleted, amplified and/or overexpressed in tumors, particularly breast cancer. The invention also provides a plurality of said genes or genetic loci that can be employed in the prognostic methods of the invention individually or in combination to effect a prognosis, more particularly a therapeutic prognosis and most particularly a clinical decision regarding chemotherapy and chemotherapeutic drug choices for an individual patient.

The invention therefore provides methods for individualized, genetic-based medicine by informing a caregiver of the likelihood of successful treatment of an individual patient with a treatment modality.

The methods of the invention are preferably performed using human cancer patient tumor samples, most preferably samples preserved, for example in paraffin, and prepared for histological and immunohistochemical analysis.

For the purposes of this invention, the term “tumor sample” is intended to include resected solid tumors, biopsy material, pathological specimens, bone marrow aspirates, and blood samples comprising neoplastic cells of hematopoietic origin, as well as benign tumors, particularly tumors of certain tissues such as brain and the central nervous system. Most particularly, the tumor samples of this invention are breast cancer tumor samples, ovarian tumor samples, or non-small cell lung (NSCLC) tumor samples.

Also included in the tumor samples of the invention are samples that have been treated or manipulated after resection to increase the proportion of tumor cells in the sample. Examples of such treatments include physical and/or enzymatic dissociation of the tumor sample and differential recovery or separation of the tumor cells from non-tumor cells (such as stromal cells, hematopoietic cells, or non-tumor tissue cells resulting, inter alia, from resection at the margins of the tumor). Tumor cell separation can be achieved using differential growth methods (in culture or in semisolid medium, for example) or by specifically or differentially labeling tumor cells and separating them thereby. For example, detectably-labeled immunological reagents (including antibodies, particularly monoclonal antibodies, or immunospecific fragments thereof) are used specifically or differentially label tumor cells, which are then separated from non-tumor cells on the basis of their specific or differential labeling. Detectable labels include, for example, fluorescent, antigenic, radioisotopic or biotin labels, among others. Alternatively, labeled secondary or tertiary immunological detection reagent can be used to detect binding of the neoplastic immunological reagents (i.e., in secondary antibody (sandwich) assays). Separation methods include, for example, immunoaffinity columns, immunomagnetic beads, fluorescence activated cell sorting (FACS), most preferably FACS.

Examples of immunological reagents useful in the practice of particular aspects of this invention include antibodies, most preferably monoclonal antibodies, that recognize tumor antigens, including but not limited to CA15-3 (breast cancer), CA19-9 (gastrointestinal cancer), CA125 (ovarian cancer), CA242 (gastrointestinal cancer), p53 (colorectal cancer), prostate-specific acid phosphatase (prostate cancer), Rb (retinoblastoma), CD56 (small cell lung cancer), prostate-specific antigen (PSA, prostate cancer), carcinoembryonic antigen (CEA), melanoma antigen and melanoma-associated antigens (melanoma), mucin-1 (carcinoma), HER2 (breast cancer), and EGFR (breast and ovarian cancer). Preferred immunological reagents recognize breast cancer, including but not limited to CA15-3, HER2 and EGFR.

The immunological reagents of the invention are preferably detectably-labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques (Maino et al., 1995, Cytometry 20: 127-133).

As used herein, the terms “microarray,” “bioarray,” “biochip” and “biochip array” refer to an ordered spatial arrangement of immobilized biomolecular probes arrayed on a solid supporting substrate. Preferably, the biomolecular probes are immobilized on second linker moieties in contact with polymeric beads, wherein the polymeric beads are immobilized on first linker moieties in contact with the solid supporting substrate. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample, either RNA or DNA. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors). Useful microarrays for detecting differential gene expression between chemotherapeutic drug sensitive and resistant neoplastic cells are described, inter alia, in U.S. Pat. No. 6,040,138 to Lockhart et al. (commercially-available from Affymetrix, Inc., Santa Clara, Calif.) and U.S. Pat. No. 6,004,755 to Wang (commercially-available from Incyte Inc., Palo Alto, Calif.) and are also commercially available, inter alia, from Research Genetics (Huntsville, Ala.). A non-limiting example of commercially available biochips useful in the practice of the invention is the Affymetrix GeneChip® Human Genome U133 Set (which includes both HG-U133A and HG-U133B). A non-limiting example of commercially available biochip in the practice of the invention is the Spectral Genomics Spectral Chip™ 2600. Arrays have been used for detecting differential copies of DNA in tumor cells are described, inter alia, in U.S. Pat. No. 6,048,695 to Bradley et al. (commercially available from Spectral Genomics Inc, Houston, Tex.).

As used in certain aspects of the methods of the invention, gene arrays or microarrays comprise of a solid substrate, preferably within a square of less than about 10 microns by 10 microns on which a plurality of positionally-distinguishable polynucleotides are attached. These probe sets can be arrayed onto areas of up to 1 to 2 cm², providing for a potential probe count of >30,000 per chip. The solid substrate of the gene arrays can be made out of silicon, glass, plastic or any suitable material. The form of the solid substrate may also vary and may be in the form of beads, fibers or planar surfaces. The sequences of these polynucleotides are determined from tumor-specific gene sets identified by analysis of gene expression profiles from a plurality of tumors as described above. The polynucleotides are attached to the solid substrate using methods known in the art (see, for example, DNA MICROARRAYS: A PRACTICAL APPROACH, Schena, ed., Oxford University Press: Oxford, UK, 1999) at a density at which hybridization of particular polynucleotides in the array can be positionally distinguished. Preferably, the density of polynucleotides on the substrate is at least 100 different polynucleotides per cm², more preferably at least 300 polynucleotides per cm². In addition, each of the attached polynucleotides comprises at least about 25 to about 50 nucleotides and has a predetermined nucleotide sequence. Larger nucleotides generated from BACs can be also be used and each of these has a predetermined sequence that is complementary to human genomic DNA. Target RNA, cDNA, or DNA preparations are used from tumor samples that are complementary to at least one of the polynucleotide sequences on the array and specifically bind to at least one known position on the solid substrate.

Gene expression analysis is performed to detect differences in gene expression between neoplastic cells that are sensitive to a cytotoxic, chemotherapeutic drug such as taxane and drug resistant neoplastic cells. RNA from the drug resistant neoplastic cells and drug sensitive neoplastic cells is individually isolated and cDNA prepared therefrom. In preferred embodiments, the cDNA is detectably labeled, for example using radioactively-labeled or fluorescently-labeled nucleotide triphosphates. Hybridization of gene expression microarrays produces patterns of gene expression specific for cytotoxic, chemotherapeutic drug resistant neoplastic cells and neoplastic cells sensitive to the same drug. Identification of genes and patterns of genes differentially expressed in these cells is established by comparison of the gene expression pattern obtained by performing the microarray hybridization analysis on cDNA from neoplastic cells that are resistant to and sensitive to the cytotoxic, chemotherapeutic drug. Advantageously, tumor samples from human patients and taxane-resistant and -sensitive breast cancer cell lines are compared using bioinformatics analysis to identify genes statistically correlated with drug resistance or sensitivity.

Once identified, differentially amplified and/or overexpressed genes can be used alone or in combination to assay individual tumor samples and determine a prognosis, particularly a prognosis regarding treatment decisions, most particularly regarding decisions relating to treatment modalities such as chemotherapeutic treatment.

Comparative Genomic Hybridization (CGH) is a technique for detecting mutations at the chromosomal level. Genomic DNA is purified from cells and labeled with fluorescent dyes. The labeled DNA is then hybridized to immobilized bacterial artificial chromosomes (BACs). BACs are artificially constructed chromosomes made from bacterial DNA and include inserted segments of 100,000-300,000 base pairs from human DNA. The resulting signals from the hybridization are analyzed for alterations in DNA gains and losses as compared to a standard human genome.

Once identified, differentially amplified or deleted genes or genetic loci can be used alone or in combination to assay individual tumor samples and determine a prognosis, particularly a prognosis regarding treatment decisions, most particularly regarding decisions relating to treatment modalities such as chemotherapeutic treatment. These changes can be detected with such procedures as Fluorescence In Situ Hybridization (FISH), Chromogenic In Situ Hybridization (CISH), or CGH arrays.

The methods of the invention for identifying genes or genetic loci deleted, amplified and/or overexpressed by drug resistant tumor cells comprise comparing the levels of drug resistance as determined by EDR® assay with differential gene amplification and overexpression. Gene expression levels as determined by gene array hybridization are compared between tumor samples that are extreme drug resistant versus low (or intermediate) drug resistant, and a comparator ratio established. It has been estimated in the art that a four-fold level of gene amplification corresponds to at least a 1.2-fold increase in gene expression. Therefore, the ratio of gene expression level for any specific drug-resistance-related gene amplification in the comparison between low drug resistant tumors and extreme drug resistant tumors as determined by EDR® assay is preferably at least 1.2, and more preferably at least 2. The ratio of gene expression level for any specific drug-sensitivity-related gene amplification in the comparison between extreme drug resistant tumors and low drug resistant tumors as determined by EDR® assay is preferably at least 1.2, and more preferably at least 2.

Once genes or genetic loci showing differential expression between low drug resistant tumors and extreme drug resistant tumors as determined by EDR® assay at the chosen ratio, genes that have a high frequency of “absence” (as indicated by the Affymetrix software for gene expression) or genetic loci that lack a statistically significant variation from the control (as calculated by Spectral Ware on CGH arrays) are excluded. Such genes or genetic loci include genes or genetic loci showing differential expression due to cross-hybridization to other genes or genetic loci, or where hybridization occurs between both exact matches and single mismatches, according to the quality control oligonucleotides included within the Affymetrix U133A and U133B gene array. Similarly, duplicate genes contained in the gene array are excluded (i.e., a gene is only scored once in the differentially-expressed gene set). The resulting gene set should contain all the genes overexpressed in extreme drug resistance-exhibiting tumor samples compared with low drug resistance-exhibiting tumor samples as determined by EDR® assay at the selected comparator ratio.

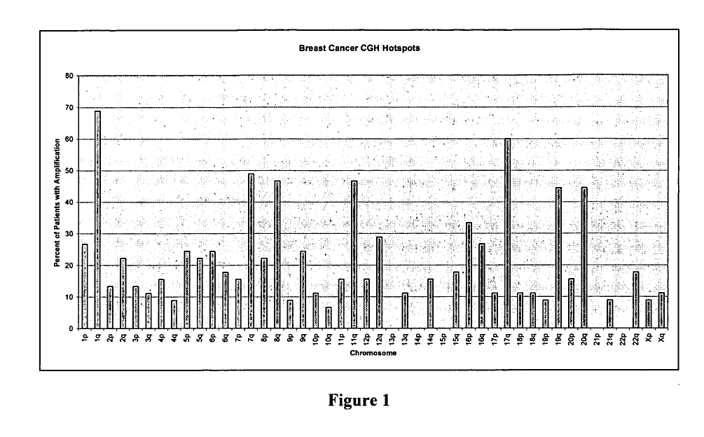

This gene set is then sorted by chromosomal location, using a database of gene locations. Most preferably, the database is a high fidelity chromosomal map, such as the University of California at Santa Cruz map at http://genome.ucsc.edu (last visited Nov. 11, 2004). The sorted gene set is then analyzed to detect regions where genes or genetic loci from adjacent or proximal loci were coordinately overexpressed, suggesting that the chromosomal region of these loci were amplified in extreme drug resistance tumor samples. Said cohorts of sorted and co-overexpressed loci are then compared to a map of chromosomal regions amplified in tumor samples. An example of such a map is shown in FIG. 1, which displays a histogram of percentage of amplified chromosomal loci in breast cancer tumor samples. Restricting the differentially-expressed gene set to genes at amplified genetic loci increases the likelihood that overexpression is related to chromosomal amplification rather than merely overexpression of genes through increased transcription rate of non-amplified genes. To further improve the likelihood that the correlation between gene overexpression and DNA amplification is related to drug resistance, a threshold cut-off for the percentage of tumor samples showing DNA amplification that correlates with overexpression of genes residing at the amplified chromosomal location is chosen. As provided here, that threshold is determined by an assessment of the lowest value that reliably correlates gene amplification and overexpression of genes at the amplified chromosomal loci.

Finally, genes or genetic loci in the sorted gene set are excluded if their expression levels are known to be coordinately regulated, such as genes sharing known enhancer/repressor regulatory sequences.

An explicit, non-limiting example of the methods of the invention for identifying differentially-expressed genes relating to drug resistance is provided herein for the chemotherapeutic drug paclitaxel. Paclitaxel is used extensively for treating breast cancer, and breast cancer tumor samples were subjected to EDR® assay using paclitaxel. As disclosed more fully in the Examples below, the practice of the inventive methods identified five human genes as being amplified and overexpressed in paclitaxel-resistant breast cancer tumor samples, each residing on chromosome 1q:
H2B histone family, member Q (H2BFQ, Genebank Accession No. BC005827)
Soluble carrier family 19 (thiamine transporter) member 2 (SLC19A2, Genebank Accession No. AJ237724)
Zinc finger protein 281 (ZNF281, Genebank Accession No. BC060820)
Decay accelerating factor for complement (CD55/DAF, Cromer blood group system; Genebank Accession No. BT007159)
Activating transcription factor 3 (ATF3, Genebank Accession No. BC006322)

Identification of these genes provides reagents and methods for providing a prognosis to improve decision-making for a clinician to choose a treatment modality for an individual patient. In the practice of one aspect of the prognostic methods of this invention, breast cancer tumor samples are obtained and analyzed for gene expression levels of one or a plurality of these genes compared with known gene expression levels for low drug resistance and extreme drug resistance tumors as determined by EDR® assay. In preferred embodiments, the genes tested for overexpression (i.e., RNA or protein over-expression) are H2BFQ, SLC19A2, ZNF281, CD55/DAF or ATF3, wherein one or a plurality of said genes is tested. Testing for overexpression of RNA is performed using any of a number of quantitative RNA technologies, including but not limited to, gene expression arrays, reverse transcription-polymerase chain reaction (RT-PCR), and Northern blotting. Testing for protein overexpression is performed using any of a number of quantitative or semi-quantitative technologies, including but not limited to, western blot, ELISA, immunohistochemistry and mass spectroscopy.

Genomic DNA from a tumor sample, most preferably a human tumor sample, can also be assayed to detect gene amplification of one or a plurality of these genes. Detection of gene amplification and/or overexpression of any or a plurality of these genes in a tumor sample indicates that paclitaxel is a clinically-inappropriate treatment modality for the human patient who provided the sample. On the other hand, failure to detect gene amplification and/or overexpression of any or a plurality of these genes in a tumor sample indicates that paclitaxel can be a clinically-appropriate treatment modality for the human patient, that can be initiated with higher confidence than without the genetic information provided by the methods of this invention. Moreover, residual tumor can be assayed during and throughout the course of treatment, to detect development of paclitaxel resistance, inter alia, by selection of a subpopulation of tumor cells having gene amplification and/or overexpression of any or a plurality of these genes.

The most direct method to discover genetic loci that vary between drug resistant and drug sensitive tumors is through CGH screening. Purification of DNA is done using any of several commercially available kits (such as those produced by Qiagen, Chatsworth, Calif. and Promega, Madison, Wis.). The DNA from the sample and DNA from a reference are labeled with Cy3 and Cy5 labelled nucleotides via nick translation, one dye for each strand, and hybridized to a CGH array, for example, made up of BACs covering the entire human genome. The signal from the hybridized and labeled DNA is efficiently quantitated by software analyses, for example SpectralWare by Spectral Genomics (Houston, Tex.). This software compares the intensity of the signal from both dyes and the signals from the BACs that encode DNA located near the target in the human genome. The significance of the change is then incorporated into the reading and summarized in a report. These analyses were done for each tumor and each chromosome in the examples set forth below, and generally are the most effective manner of detecting chemotherapeutic drug treatment-related genetic amplification. Genetic loci described herein were found to be either amplified or deleted in four of the sixteen Taxol EDR tumors and in one or less of the Taxol LDR tumors.

Amplification of DNA from a chromosomal region identified as described herein, or RNA or protein overexpression from genes or genetic loci in these chromosomal regions may be used to predict resistance to paclitaxel therapy and consequent poor prognosis in multiple tumor types, including breast cancer, ovarian cancer, and non-small cell lung cancer. In preferred embodiments, the chromosomal regions amplified in paclitaxel resistant tumors are human chromosome 1q21-1q25, 1q32, 1q41, 1q42, 1q43, 1q44, 2q31, 6p22-25, 8q12, 14q32, and 20q13. In the preferred embodiments, the chromosomal regions that are deleted in paclitaxel resistant tumors are human chromosomes 2q12, 4p12-15, 4q27-31, 5q23, 6q26, 8p11-23, 11q25, 13q14, 14q12, 15q23-25, 16p11-13, and 18q23. These changes in DNA can be used to determine paclitaxel resistance in breast cancer, ovarian cancer, or non-small cell lung cancer.

For the sixteen tumors that were EDR to paclitaxel, two were EDR for docetaxel as well. Thus in a preferred embodiment, these genetic loci can be used to predict response to both paclitaxel and docetaxel. Testing for DNA amplification, preferably at these genetic loci, can be performed using any of a number of quantitative DNA technologies, including but not limited to, gene arrays, fluorescence in situ hybridization (FISH), Southern blot, polymerase chain reaction (PCR), enzyme-linked immunosorbent assay (ELISA) and comparative genomic hybridization (CGH).

As provided herein, the methods of this invention are directed towards determining resistance of a tumor sample, most preferably a human tumor sample, to chemotherapeutic drugs. In certain embodiments, these drugs are microtubule binding and stabilizing agents, including but not limited to paclitaxel, docetaxel and epothilones, and more specifically paclitaxel. The description set forth above and the Examples set forth below recite exemplary embodiments of the invention. However, the disclosure set forth herein is intended to encompass any chemotherapeutic drug that induces amplification or deletion of genes or genetic loci, and any tumor sample, most preferably any human tumor sample, comprising cells whose chromosomal DNA comprises amplified or deleted genes or genetic loci.

The following Examples are intended to further illustrate certain preferred embodiments of the invention and are not limiting in nature.

### EXAMPLES

### Example 1

Tumor Specimen Handling

Viable tumors samples were obtained from patients with malignant disease and placed into Oncotech transport media (complete medium, RPMI supplemented with 3% Fetal Calf Serum and antibiotics, as described below in the section Tissue Culture and Expansion) by personnel at the referring institution immediately after collection and shipped to Oncotech by overnight courier for the purpose of determining the tumors in vitro drug response profile. Upon receipt, data on tissue diagnosis, treatment history, referring physician, and patient information about the specimen was entered into a computer database. The tumor was then processed by the laboratory where three areas of the tumor are removed from the sample, fixed in Formalin, paraffin embedded, sectioned and hematoxylin and eosin stained for pathologists' review to ensure agreement with the referring institution histological diagnosis. After in vitro drug response of the tumor specimens were determined by the laboratory, this information was sent back to the treating physician to aid in their treatment selection.

The remainder of the sample is disaggregated mechanically and processed into a cell suspension for the Extreme Drug Resistance (EDR) assay. A cytospin preparation from a single cell suspension of the tumor was examined by a technologist to determine the presence and viability of malignant cells in the specimen.

EDR Assay

The EDR assay is an agarose-based culture system, using tritiated thymidine incorporation to define in vitro drug response. This assay is predictive of clinical response (Kern et al., 1990, “Highly specific prediction of antineoplastic resistance with an in vitro assay using suprapharmacologic drug exposures,” J. Nat. Cancer Inst. 82: 582-588). Tumors were cut with scissors into pieces of 2 mm or smaller in a Petri dish containing 5 mL of complete medium. The resultant slurries were mixed with complete media containing 0.03% DNAase (2650 Kunitz units/mL) and 0.14% collagenase I (both enzymes obtained from Sigma Chemical Co., St. Louis, Mo.), placed into 50 mL Erlenmeyer flasks with stirring, and incubated for 90 min at 37° C. under a humidified 5% CO₂ atmosphere. After enzymatic dispersion into a near single cell suspension, tumor cells were filtered through nylon mesh, and washed in complete media. A portion of the cell suspension was used for cytospin slide preparation and stained with Wright-Giemsa for examination by a medical pathologist in parallel with Hematoxylin-Eosin stained tissue sections to confirm the diagnosis and to determine the tumor cell count and viability. Tumor cells were then suspended in soft agarose (0.13%) and plated at 20,000-50,000 cells per well onto an agarose underlayer (0.4%) in 24-well plates. Tumor cells were incubated under standard culture conditions for 4 days in the presence or absence of 2.45 μM paclitaxel. Cells were pulsed with tritiated thymidine (New Life Science Products, Boston, Mass.) at 5 μCi per well for the last 48 hours of the culture period. After labeling, cell culture plates were heated to 96° C. to liquify the agarose, and the cells are harvested with a micro-harvester (Brandel, Gaithersburg, Md.) onto glass fiber filters. The radioactivity trapped on the filters was counted with an LS-6500 scintillation Counter (Beckman, Fullerton, Calif.). Untreated cells served as a negative control. In the positive (background) control group, cells were treated with a supratoxic dose of Cisplatin (33 μM), which causes 100% cell death. Detectable radioactivity for this group was considered non-specific background related to debris trapping of tritiated thymidine on the filter. After subtracting background control values, percent control inhibition (PCI) of proliferation was determined by comparing thymidine incorporation by the treatment group with incorporation by the negative control group: PCI=100%×[1−(CPM treatment group/CPM control group)]. The determinations of drug effects on tumor proliferation was performed in duplicate. Specimens were classified as EDR to paclitaxel if the PCI was 19% or less. Specimens were classified as LDR to paclitaxel if the PCI was 43% or greater.

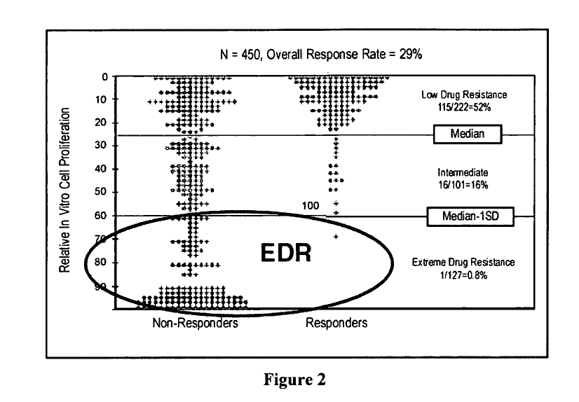

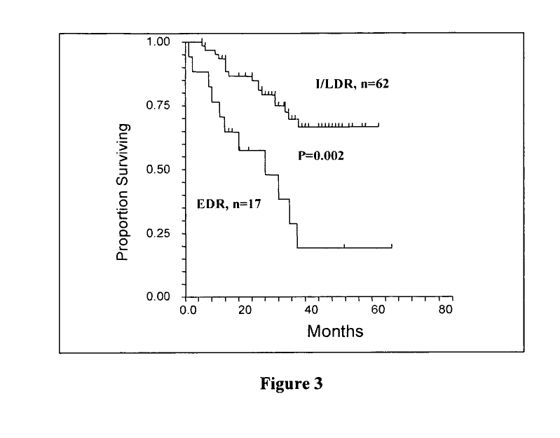

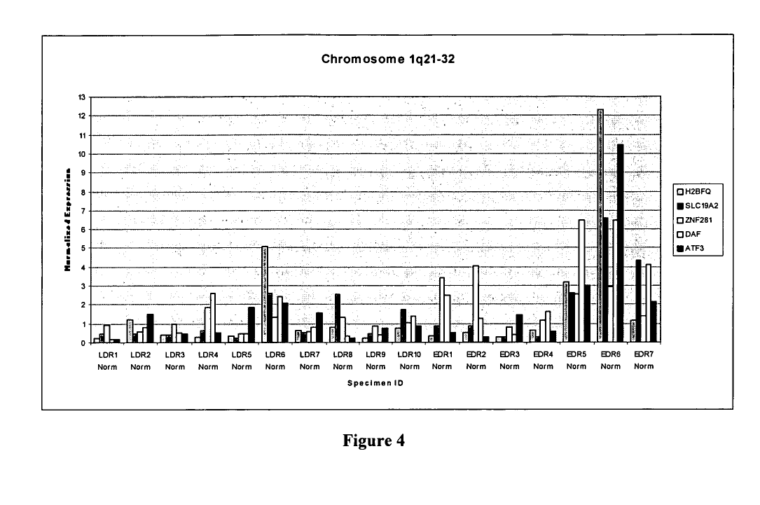

The results of EDR® assay on breast cancer tumor samples is shown in FIG. 2, and the correlation between survival and drug resistance as determined by the assay are shown in FIG. 3.

### Example 2

Gene Array Analyses

Neoplastic cells from breast cancer specimens defined as EDR or LDR to paclitaxel were used to make mRNA for performing gene array hybridization analyses. Some cells were expanded by growth in culture in vitro and/or were differentially cultured or sorted by flow cytometric sorting to increase the number and percent of tumor cells.

RNA Isolation

Cells were thawed and pelleted by centrifugation at 12,000×g for 5 minutes in a clean 1.5 mL eppendorf tube. The supernatant was discarded with care not to disturb the pellet. One mL of TRIzol® reagent (Invitrogen Life Technologies, Carlsbad, Calif.) was added per 5 million cells, and the cells lysed by repetitive pipetting. Insoluble material was removed from the homogenate by centrifugation at 12,000×g for 10 minutes.

The cleared homogenate solution was transferred to a fresh 1.5 mL tube and the homogenized samples were incubated for 5 minutes at 15 to 30° C. to permit the complete dissociation of nucleoprotein complexes. Two hundred microliters of chloroform were added per 1 mL of TRIzol® reagent, the tubes capped securely and shaken vigorously by hand for 15 seconds. After shaking, the tubes were incubated at 15 to 30° C. for 2 to 3 minutes and then centrifuged at 12,000×g for 15 minutes.

The upper aqueous phase containing the RNA was transferred to a clean 1.5 mL eppendorf tube. Five hundred microliters of isopropyl alcohol was added per 1 mL of TRIzol® reagent used for the initial homogenization to precipitate RNA from the aqueous phase. Samples were incubated at 15 to 30° C. for 10 minutes and centrifuge at 12,000×g for 10 minutes. The supernatant was discarded with care not to disturb the gel-like RNA pellet. The RNA pellet was then washed with 75% ethanol, pipetting at least 1 mL of 75% ethanol per 1 mL of TRIzol® Reagent used for the initial homogenization. The sample was mixed by vortexing and centrifuged at 6,000×g for 5 minutes, and the RNA pellet briefly dried at 15 to 30° C. for 5 to 10 minutes. One hundred microliters of RNase-free water was added and the RNA was resuspended by passing the solution through a pipette tip. To aid in resuspension, the sample was incubated at 55 to 60° C. for 10 minutes.

Specimens were then further purified using RNeasy® spin columns (obtained from Qiagen Corporation, Valencia, Calif.) using a protocol supplied by the manufacturer. Briefly, 350 μL Buffer RLT (containing beta-mercaptoethanol) was added and mixed thoroughly. Absolute ethanol (250 μL) was added to the diluted RNA and mixed thoroughly by pipetting. The sample (700 μL total) was added to an RNeasy® mini column placed in a clean 2 mL collection tube (supplied with RNeasy® Mini Kit), and centrifuged for 15 to 20 seconds at 8,000×g. The sample was re-pipetted onto the RNeasy® mini column and again centrifuged for 15 to 20 sec at 8,000×g. The flow-through was discarded. The column was washed using 350 μL Buffer RW1 that was added to the RNeasy® mini column, centrifuged for 15 sec at 8000×g, and again the flow-through discarded. This washing step was repeated, and then 500 μL Buffer RPE (with ethanol added) was added to the RNeasy® mini column and the column centrifuged for 15 to 20 sec at 8,000×g.

For eluting the RNA from the column, the RNeasy® column was transferred into a clean 2 mL collection tube and 500 μL Buffer RPE was added and centrifuged for 2 to 3 minutes at 8,000×g to dry the RNeasy® silica-gel membrane. The RNeasy® column was transferred into a clean 1.5 mL collection tube and 30 μL RNase-free water was added directly onto the RNeasy® column without disturbing the silica-gel membrane. The water was allowed to soak into the silica-gel membrane for 1 minute and the column was then centrifuged for 2 to 3 minutes at 8,000×g to elute the RNA. An aliquot was removed for UV spectroscopy and QC purposes, and the RNA specimens were stored at −70° C. or colder until use.

The yield and purity of total RNA was determined spectrophotometrically. Specimen concentration and purity was determined by absorbance of ultraviolet light at 260 and 280 nm. The A₂₆₀/A₂₈₀ ratio was calculated and the concentration of RNA was determined using Beers law. Specimen integrity was analyzed by agarose-based electrophoresis and pictures were recorded. The integrity of the RNA was evaluated by checking the quality of the 28S and 18S ribosomal RNA peaks and the ratio thereof. While determination of RNA integrity is somewhat subjective, a 2:1 ratio of 28S to 18S RNA is considered excellent.

MicroArray Assay

cDNA Synthesis and Gene Expression Profiling. Total RNA (5 to 15 μg) was used to generate double-stranded cDNA by reverse transcription using a cDNA synthesis kit (Superscript Choice System, Life Technologies, Inc., Rockville, Md.) that uses an oligo(dT)₂₄ primer containing a T7 RNA polymerase promoter 3′ to the poly T (Geneset, La Jolla, Calif.), followed by second-strand synthesis. Labeled cRNA was prepared from the double-stranded cDNA by in vitro transcription by T7 RNA polymerase in the presence of biotin-11-CTP and biotin-16-UTP (Enzo, Farmington, N.Y.). The labeled cRNA was purified over RNeasy® columns (Qiagen, Valencia, Calif.). Fifteen μg of cRNA was fragmented at 94° C. for 35 minutes in 40 mmol/L of Tris-acetate, pH 8.1, 100 mmol/L of potassium acetate, and 30 mmol/L of magnesium acetate. The cRNA was then used to prepare 300 μL of hybridization cocktail (100 mM MES, 1 mol/L NaCl, 20 mM ethylenediaminetetraacetic acid, 0.01% Tween 20) containing 0.1 mg/mL of herring sperm DNA (Promega, Madison, Wis.) and 500 μg/mL of acetylated bovine serum albumin (Life Technologies, Inc.).

Before hybridization, the cocktails were heated to 94° C. for 5 minutes, equilibrated at 45° C. for 5 minutes, and then clarified by centrifugation (16,000×g) at room temperature for 5 minutes. Aliquots of this hybridization cocktail containing 15 μg of fragmented cRNA were hybridized to Test3 chip arrays or U133A and U133B arrays at 45° C. for 16 hours in a rotisserie oven at 60 rpm. After hybridization, the gene chips are automatically washed and stained with streptavidin-phycoerythrin using a fluidics station as follows: the arrays are washed using nonstringent buffer (6×SSPE) at 25° C., followed by stringent buffer (100 mmol/L MES, pH 6.7, 0.1 mol/L NaCl, 0.01% Tween 20) at 50° C. The arrays were stained with streptavidin-phycoerythrin (Molecular Probes, Eugene, Oreg.), washed with 6× sodium chloride, sodium phosphate, EDTA (SSPE buffer), incubated with biotinylated anti-streptavidin IgG, stained again with streptavidin-phycoerythrin, and washed again with 6×SSPE. The arrays were scanned using the GeneArray scanner (Affymetrix). Image analysis was performed with GeneChip software (Affymetrix).

The Test3 chip array contained 24 individual housekeeping genes with representative segments spanning the 5′, middle and 3′ portions of the genes. These probe sets made it possible to confirm that the cRNA synthesis covered the entire length of the transcripts present, and that it was not degraded. Halting a run of U133A and U133B chips based on poor test chip results offers a substantial quality control measure to the experimental process. If the test chip results passed the quality control criteria, the labeled cRNA was applied to the U133A or U133B arrays. The arrays were scanned at 3-mm resolution using the Genechip System confocal scanner made for Affymetrix by Agilent (Agilent G2565AA DNA microarray scanner). Microarray Suite 5.1 software from Affymetrix was used to determine the relative abundance of each gene, based on the average difference of intensities. Data analysis began with scanning, which collected data for each feature, containing an identical sequence set of 25-mers in an 18 μm area. Each feature was scanned 6 times to collect a 6×6 set of pixels covering the 18 μm area. Only the inner set of 4×4 pixels were read as the probe pixel set to avoid collection of signal bleed from adjacent elements. The chip was segmented into 16 zones, and a background correction was applied by subtracting the lowest 2% of signal values calculated for these zones adjusted by a distance weighting such that the local background within a zone contributed more heavily to the 2% calculation than do more distant zones. Thus, each zone had its own unique 2% background correction value. After background correction, the 16 signal values for each reading set were arranged into a normal distribution, and the signal value that fell at the 75^{th} percentile was selected as the final feature signal. These data were collected in a file. The raw output of the scanned image was visually inspected prior to further data analysis to assure that no fractures in the chip surface occurred during processing, and that the signal strength was uniform on the chip.

### Example 3

Determination of Drug-Resistance Specific Gene Amplification Data Set

A multi-step algorithm was employed to identify regions of amplified DNA in breast cancer tumor samples using Gene Chip RNA expression data. As described above, breast cancer specimens were defined initially as intrinsically resistant (EDR) or intrinsically sensitive (LDR) to paclitaxel using the EDR® assay described in Example 2 above (and commercially available from Oncotech, Inc., Tustin, Calif.). A gene expression ratio of all genes profiled on the Gene Chip was determined by dividing the mean gene expression signal for paclitaxel EDR specimens by the mean gene expression signal for paclitaxel LDR specimens for each gene in the array. It was determined that a ratio of at least 2.2 was required to indicate that the gene occupied a genetic locus at a chromosomal region where DNA amplification may relate to paclitaxel resistance.

After having made this determination, the data set was refined by removing gene array data deemed unreliable based on a high “frequency of absence” as called by the expression analysis software supplied by the gene chip manufacturer (Affymetrix). Genes or genetic loci remaining in the data set were then sorted by chromosomal location using publicly-available high fidelity chromosomal map location data (as is available, inter alia, at the UCSB genome website, genome.ucsc.edu). The genes or genetic loci that showed significantly-increased expression in paclitaxel EDR specimens were then arranged by chromosomal location. From this set of identified genes or genetic loci, candidate genes or genetic loci or genomic loci were selected based on the frequency of coordinately-increased gene expression in the same chromosomal region. Paclitaxel EDR specimens showing increased mean gene expression compared to the mean expression level in paclitaxel LDR specimens were considered potentially amplified. If these specimens also contained significantly increased gene expression of adjacent genes, the likelihood of gene amplification was also increased (in contrast,for example, to non-copy number related expression increases).

For each gene or genetic locus selected to be part of the gene set, said gene or genetic locus was expressed or coordinately amplified in at least 40% of tumor samples determined to be EDR. This was done to ensure sufficiently high sensitivity for the assay to provide clinically-relevant results. The data was further evaluated for any relationship to known regions of chromosomal amplification, since such evidence would further support the link between increased gene expression and genomic amplification.

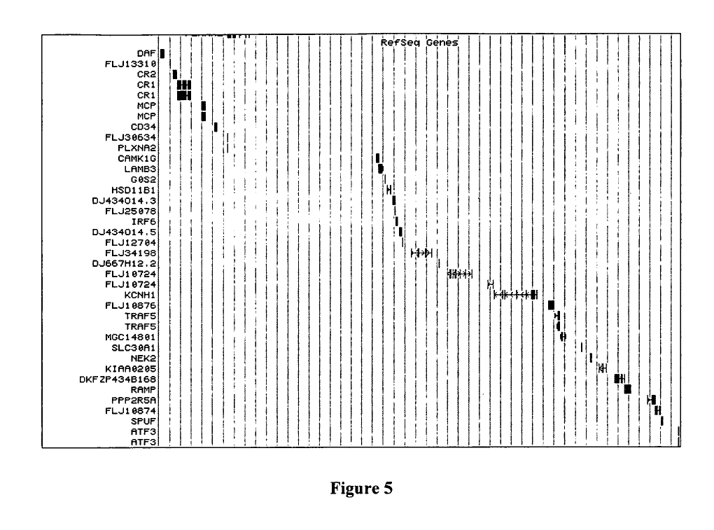

The results of these determinations are shown in FIG. 5, showing regions of chromosome 1q21-23 and 1q32 that are amplified in greater than 50% of breast cancers. The genes in these regions do not have shared metabolic or pathway functions and are unlikely to be coordinately regulated (for example, as the result of known shared enhancer/repressor sequences). Five genes from these regions (H2BFQ, SLC19A2, ZNF281, DAF and ATF3) were chosen for use in the assays of the invention, as described in additional detail above.

TABLE I

| Gene Name | Abbrev | Location | High Resolution Location |
|---|---|---|---|
| H2B histone | H2BFQ | 1q21-q23 | chr1: 145598444-145600666 |
| family, member | | | |
| Q | | | |
| solute carrier | SLC19A2 | 1q23.3 | chr1: 165084004-165105999 |
| family 19 | | | |
| (thiamine | | | |
| transporter), | | | |
| member 2 | | | |
| zinc finger | ZNF281 | 1q32.1 | chr1: 195834256-195837277 |
| protein 281 | | | |
| decay | DAF | 1q32 | chr1: 203205265-203244066 |
| accelerating | | | |
| factor for | | | |
| complement | | | |
| (CD55, Cromer | | | |
| blood group | | | |
| system) | | | |
| activating | ATF3 | 1q32.3 | chr1: 208625319-208637324 |
| transcription | | | |
| factor 3 | | | |

TABLE II

| Abbrev | Comments |
|---|---|
| H2B histone family, | DNA-binding protein |
| member Q | |
| Solute carrier family 19 | Involved with thiamine metabolism |
| (thiamine transporter), | |
| member 2 | |
| Zinc finger protein 281 | Little known |
| Decay Accelerating Factor | GPI-linked protein that inhibits |
| for Complement (DAF, | complement-mediated cell death. |
| CD55) | Loss of DAF in patients with PNH |
| | leads to erythrocyte lysis. Also expressed |
| | in solid tumors to varying degrees |
| Activating transcription | Mediates generalized cellular |
| factor 3 | stress responses that lead to apoptosis. May |
| | negatively regulate cell growth. May |
| | have a protective role vs. |
| | ionizing radiation. |

### Example 4

Determination of Increases in DNA Copy Number in Taxol Resistant Ovarian Tumors by PCR

To validate the gene expression data and determine if the difference in expression between Taxol resistant and Taxol sensitive tumor samples was due to an increase in DNA copy number, PCR primers were designed to the DNA sequence encoding four of the genes: IL6R (located at 1q21), H2B (located between 1q21-23), DAF (located at 1q32), and ATF3 (located at 1q32.5). The sequences of these primers were as follows:

| | | |
|---|---|---|
| 5′ IL6R: | AAGCCACAGACCCAGCAAGCAAAAG | (SEQ ID NO:1) |
| 3′ IL6R: | CTGGACGGTGCTGGGCTAGAGTGTT | (SEQ ID NO:2) |
| 5′ H2B: | CAAAAGCAAATCCAATGACGCACTG | (SEQ ID NO:3) |
| 3′ H2B: | CGTTCATTTCCATTGGTCCGTGTGC | (SEQ ID NO:4) |
| 5′ DAF: | CAGTGTCCATGCGTGAAT | (SEQ ID NO:5) |
| 3′ DAF: | GACAACTCAGCCTACATACCCAAG | (SEQ ID NO:6) |
| 5′ ATF3: | AGTGAGTGCTTCTGCCATCGTC | (SEQ ID NO:7) |
| 3′ ATF3: | AGAACGGGTAGGGATGGGGT | (SEQ ID NO:8) |

DNA from four ovarian tumors, two Taxol EDR and two Taxol LDR, were purified as follows. A commercially-available DNA Extraction kit (Gentra, Minneapolis, Minn.) was utilized to isolate intact genomic DNA from tumor explants and primary cell lines, as per manufacture's recommendation. Briefly, approximately one million cells were pelleted by centrifugation at 13,000×g for 1 minute and the media decanted. The cell pellet was vortexed and resuspended in the residual media (ca. 20 microlitre). 250 microlitre of lysis buffer (containing RNaseA and Proteinase K) was added and rapidly mixed to achieve confluent lysis and then incubates at 37 C for 30 minutes. A protein precipitation solution provided by the kit manufacturer (100 μL) was added and mixed by vortexing vigorously. Cellular debris was pelleted by centrifugation at 13,000×g for 5 minutes. The supernatant was removed to a fresh tube containing 800 μL ice cold absolute ethanol and DNA was precipitated by rocking the tube back and forth until the precipitated DNA was visualized. The precipitated DNA was removed to a fresh tube and air dried for 10 minutes, and then resuspended in an adequate volume of DNA Hydration Solution to generate 500 to 1000 μg/ml DNA (final concentration). DNA rehydration was facilitated by incubation at room temperature for an additional 60 to 120 minutes.

Ten (10) ng of DNA prepared as described above was added to a PCR mixture containing 1 μL of 5′ primer (10 μM stock), 1 μL 3′ primer (10 μM stock), 0.25 μL 10 mM dNTPs, 2 μL 5×PCR buffer (Takara Bio Inc., Shiga, Japan), 0.15 μL ExTaq, 1 μL 25 mM MgCl₂, and 2.6 μL water. The reactions were run for thirty cycles consisting of incubation at 94° for 30 seconds, incubation at 55° for 60 seconds, and incubation at 72° for 60 seconds, followed by one cycle of 94° for 30 seconds, 55° for 60 seconds, and 72° for three minutes. Five (5) μL aliquots of each PCR mixture were them run on a 1% agarose gel and stained with Ethidium Bromide.

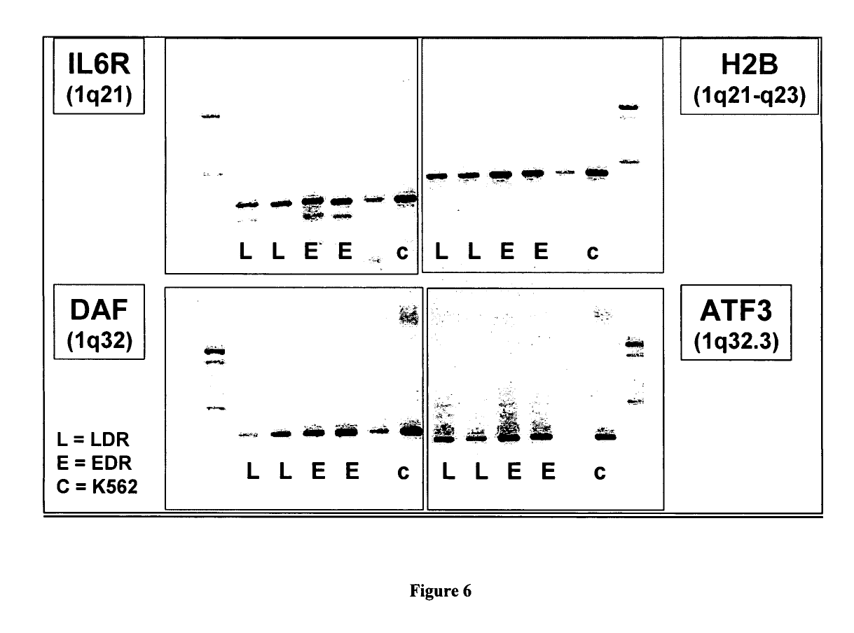

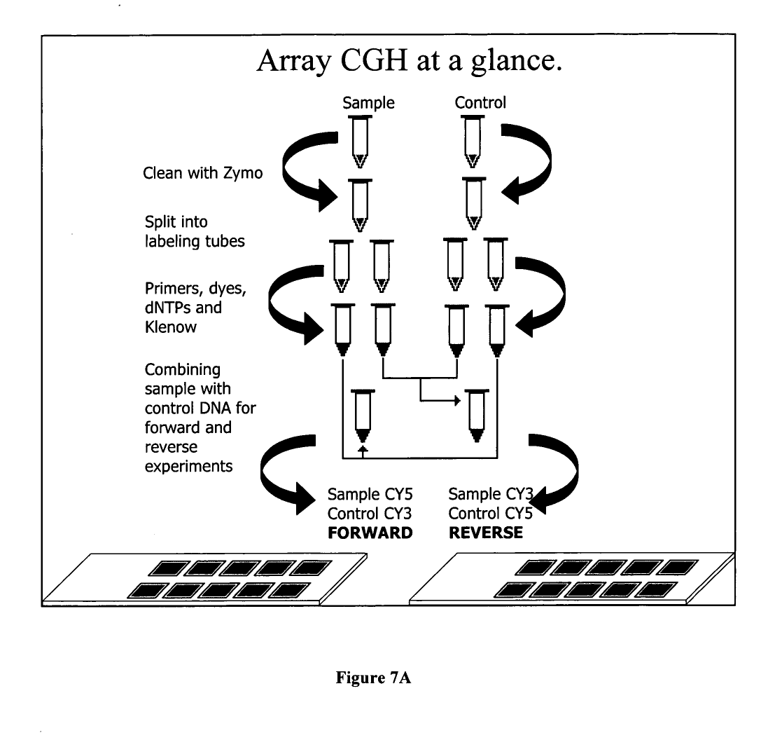

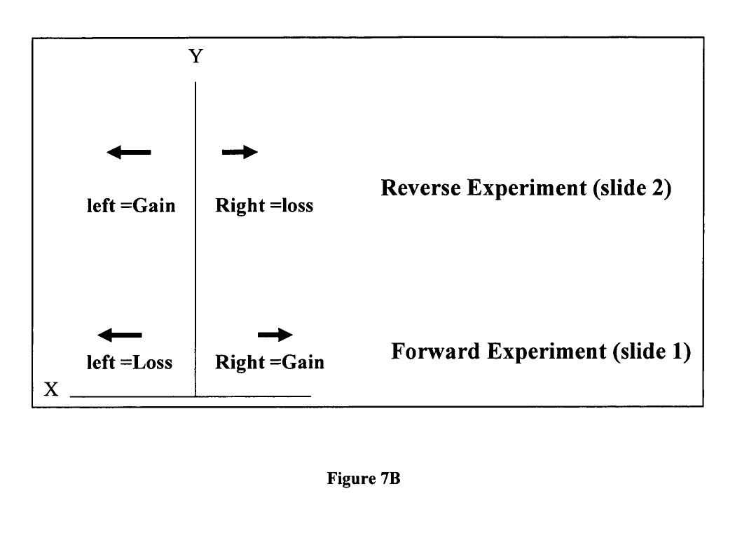

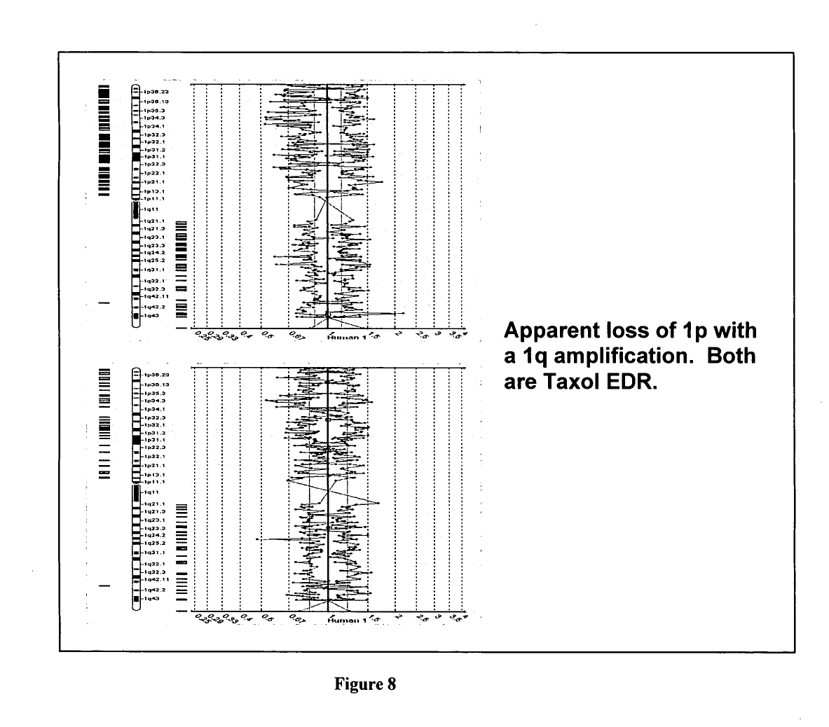

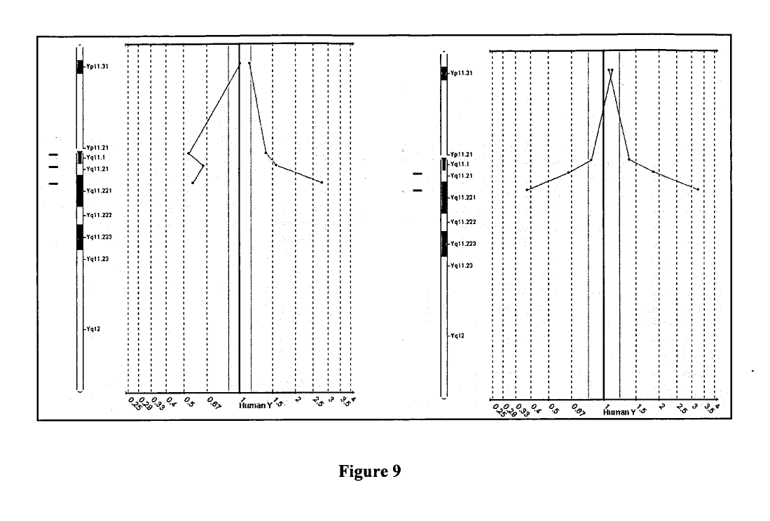

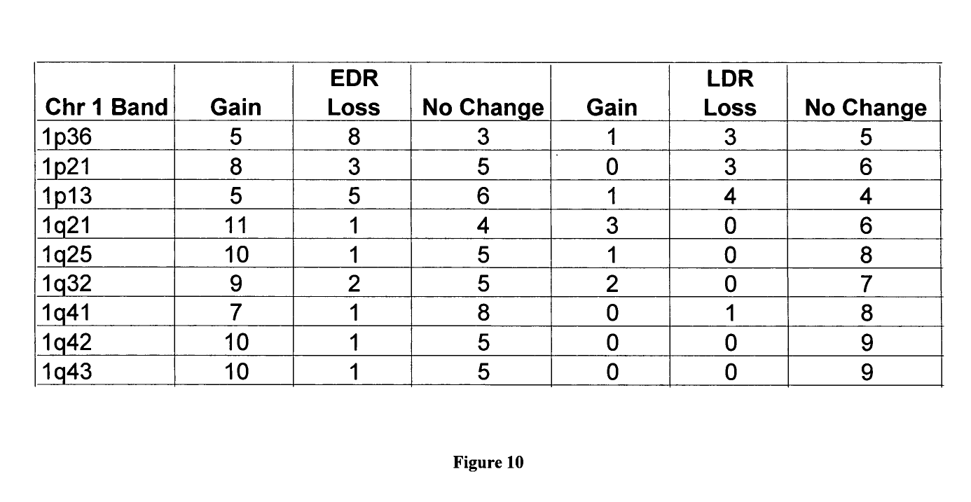

These results are shown in FIG. 6. As shown in the Figure, IL6R, H2BFQ, DAF, and ATF3 were present at higher copy number in the Taxol EDR specimens.

### Example 5

CGH Arrays for Determining Changes in DNA Copy Number.

Genomic DNA was purified from sixteen Taxol EDR ovarian tumors and six Taxol LDR ovarian tumors, using the Gentra Tissue DNA Extraction kit as described above and according to the manufacturer's recommendations. Intact genomic DNA was isolated from tumor explants and primary cell lines (approximately one million cells apiece).

All microarrays were performed utilizing Spectral Genomics' (Houston, Tex.) 1 Mb Genomic Microarrays and 1 μg of high molecular weight, RNA-free genomic DNA from fixed tumor samples. Ultra-pure deionized water was used for the preparation of all reagents; Promega Male Genomic DNA (Madison, Wis.) was used as reference DNA; and dye-reversal experiments were performed for each sample (whereby 2 microarrays, each with reciprocal labeling of the test and reference DNAs, were performed). The test and reference DNAs were random-primed labeled by combining 1 μg genomic DNA (gDNA) and water to a total volume of 50 μL and sonicating in an inverted cup horn sonicator to obtain fragments 600 bp to 10 kb in size. The DNA fragment mixture was then purified using a commercially-available kit (Zymo's Clean-up Kit, Orange, Calif.) according to the manufacturer's protocol except that final elution was performed with 2 volumes of 26 μL doubly-distilled water. The elutant was split equally between 2 tubes and, to each, 20 μL 2.5×random primers from a commercially-available DNA labeling kit (BioPrime DNA Labeling Kit, Invitrogen, Carlsbad, Calif.) was added, mixed well, boiled 5 minutes, and then immediately placed on ice for 5 minutes. To each sample tube was added 0.5 μL Spectral Labeling Buffer (Spectral Genomics), 1.5 μL Cy3-dCTP or 1.5 μL Cy5-dCTP respective to each dye-reversal experiment (PA53021, PA55021; Amersham Pharmacia Biotech, Piscataway, N.J.), and 1 μL Klenow fragment (BioPrime DNA Labeling Kit; Invitrogen). The contents were incubated for 1.5 hours at 37 C before stopping the labeling reaction by adding 5 μL 0.5 M EDTA, pH 8.0 and incubating for 10 minutes at 72° C. For hybridization to the array, the Cy3-labeled test DNA and Cy5-labeled reference DNA and, conversely, the Cy5- labeled test DNA and Cy3-labeled reference DNA were combined. Forty-five microliters human Cot-1 DNA (Invitrogen), 11.3 μL 5M NaCl, and 110 μL room temperature isopropanol were added, mixed, and allowed to stand 15 minutes before centrifugation at 13,000 rpm for 15 minutes. The pellet was washed with 500 μL 70% ethanol and allowed to air dry for 10 minutes. Onto each pellet, 50 μL hybridization solution (50% deionized formamide, 10% dextran sulfate, 2×SSC, 2% SDS, 6.6 μg/μL yeast tRNA in ultrapure water) was added and incubated for 10 minutes at room temperature before repeat pipetting to fully resuspend. The probes were denatured by incubation for 10 minutes at 72° C., then immediately placed on ice for 5 minutes. Samples were incubated at 37° C. for 30 minutes before pipetting down the center length of a 22×60-mm coverslip and placing in contact with a microarray slide. Each slide was enclosed in an incubation chamber and incubated, rocking, at 37° C. for 16 hours.

Post-hybridization washes were performed with each slide in individual deep Petri dishes in a rocking incubator. After removing the coverslip, the slides were briefly soaked in 0.5% SDS at room temperature. Each slide was then transferred quickly to 2×SSC, 50% deionized formamide pH 7.5 for 20 minutes; then 2×SSC, 0.1% IGEPAL CA-630 pH 7.5 for 20 minutes followed by 0.2×SSC pH 7.5 for 10 minutes, each solution having been pre-warmed to 50° C. and agitated in an incubator at 50° C. Finally, each slide was briefly rinsed in 2 baths of room temperature doubly-distilled water and immediately blown dry with compressed nitrogen gas and scanned.

Scanning was performed with Axon Instrument's (now, Molecular Devices Corp., Sunnyvale, Calif.) GenePix 4000B microarray scanner and the images were analyzed with SpectralWare 2.0 (Spectral Genomics, Inc.) for preparation of ratio plots. The human BAC clones were spotted onto glass slides at Spectral Genomics, Inc. by a printer using a print head with tips in a 12×1 configuration. The fluorescence intensity ratios for spots on the slide were grouped by print tip, and were spatially normalized by subtracting the print tip group median intensity ratio from each spot's intensity ratio; prior to this spatial normalization, some slides showed significant spatial bias (see, Amaratunga, 2004, Exploration and analysis of DNA microarray and protein array data, New York: John Wiley and Sons). Spots with low signal-to-noise (background) ratios were excluded. The mean intensity ratio for each clone was calculated from up to 4 remaining values (each clone was spotted twice on a slide, and the experiment was run in a dye-swap configuration). This provided control for potential Cy3/Cy5 induced labeling bias. The chromosome with minimum variance in clone intensity ratios was chosen as a “control chromosome.” A 99% confidence interval was calculated using the intensity ratios from this chromosome, and all clones were classified using this confidence interval. Clones with intensity ratios above this interval were considered amplified, and beneath this interval were considered deleted (i.e., greater or less than 50%). This method, when applied to samples with known abnormalities, provided correct classifications for 98.8% of normal clones, and 97.9% of amplified or deleted clones. The statistical significance of measurements using consecutive amplified or deleted clones was measured using the scan statistic (see, Zeschnigk et al., 2003, Bioinformatics 19:2335-42].

The data from Spectral Ware was provided in files as demonstrated below. The table includes the identification of the BAC, the genetic location of that BAC in the human genome, the Cy5 log ratio, the Cy3 log ratio, the loss (L) or gain (G) call, and comments on the result for each specific clone.

Test Sample: 102 (H05111)
Affected Chromosome(s)

| | | Cy5 rest | Cy3 rest | DNA | |
|---|---|---|---|---|---|
| Clone | Cyto Locn | log Ratios | log Ratios | Change | Comments |
| RP1-62I8 | 1p36.33 | −0.574 | 0.506 | L | Trend shows complete loss |
| RP1-283E3 | 1p36.21-1p36.33 | −0.532 | 0.366 | L | of 1p arm |
| RP1-163G9 | 1p36.2-1p36.3 | −0.355 | 0.503 | L | |
| RP3-491M17 | 1p36.2-1p36.3 | −0.604 | 0.548 | L | |
| RP11-33M12 | 1p36.31-1p36.33 | −0.626 | 0.478 | L | |
| RP3-438L4 | 1p36.2-1p36.3 | −0.637 | 0.449 | L | |
| RP4-633I8 | 1p36.21-1p36.32 | −0.215 | 0.398 | L | |
| RP11-476D13 | 1p36.21-1p36.33 | −0.594 | 0.291 | L | |
| AL031984.13 | 1p36.22 | −0.763 | 0.39 | L | |
| RP3-330O12 | 1p36.11-1p36.23 | −0.641 | 0.332 | L | |
| RP5-888M10 | 1p36.11-1p36.31 | −0.256 | 0.423 | L | |
| RP11-219C24 | 1p36.2-1p36.33 | −0.654 | 0.51 | L | |
| RP4-726F20 | 1p36.11-1p36.23 | −0.289 | 0.262 | L | |
| RP1-37C10 | 1p35.2-1p36.21 | −0.642 | 0.24 | L | |
| RP1-8B22 | 1p35.1-1p36.21 | −0.846 | 0.478 | L | |
| RP11-91K11 | 1p36.2-1p36.2 | −0.328 | 0.328 | L | |
| RP3-340N1 | 1p35-1p36.2 | −0.375 | 0.476 | L | |
| RP5-886K2 | 1p35.1-1p36.12 | −0.35 | 0.299 | L | |
| RP3-462O23 | 1p35.1-1p36.12 | −0.647 | 0.543 | L | |
| RP1-125I3 | 1p35.1-1p36.11 | −0.435 | 0.346 | L | |
| RP1-50O24 | 1p35.1-1p35.3 | −0.312 | 0.258 | L | |
| RP1-212P9 | 1p34.1-1p35 | −0.274 | 0.226 | L | |
| RP3-437I16 | 1p35.1-1p35.3 | −0.888 | 0.363 | L | |
| RP5-893G23 | 1p34.2-1p36.11 | −0.493 | 0.383 | L | |
| RP1-117O3 | 1p33-1p34.3 | −0.331 | 0.355 | L | |
| RP5-1007G16 | 1p34.2-1p35.3 | −0.7 | 0.517 | L | |
| RP1-34M23 | 1p34.3-1p36.11 | −0.915 | 0.333 | L | |
| RP4-655C4 | 1p32.3-1p34.3 | −0.698 | 0.69 | L | |
| AL033524.11 | 1p34.3 | −0.907 | 0.529 | L | |
| RP3-423B22 | 1p33-1p35.3 | −0.431 | 0.246 | L | |
| RP4-811I8 | 1p34.1-1p35.3 | −0.634 | 0.447 | L | |
| RP5-820O16 | 1p34.1-1p36.13 | −0.93 | 0.474 | L | |
| RP11-319C21 | 1p33-1p35.3 | −0.292 | 0.42 | L | |
| RP1-92O14 | 1p33-1p34.2 | −0.254 | 0.385 | L | |
| RP4-639P2 | 1p32.3-1p34.1 | −0.261 | 0.269 | L | |
| RP11-112C15 | 1p32.3-1p34.1 | −0.394 | 0.27 | L | |
| RP5-1024N4 | 1p32.1-1p33 | −0.37 | 0.256 | L | |
| RP4-814E15 | 1p32.1-1p33 | −0.268 | 0.289 | L | |
| RP5-1013G21 | 1p32.2-1p33(SC) | −0.415 | 0.533 | L | |
| RP5-965L7 | 1p32.1-1p33 | −0.339 | 0.478 | L | |
| RP11-205P11 | 1p32.3-1p33 | −0.341 | 0.283 | L | |
| RP5-879H24 | 1p32.2-1p33 | −0.684 | 0.579 | L | |
| RP4-542O18 | 1p32.3-1p34.1 | −0.517 | 0.359 | L | |
| RP11-221L2 | 1p32.2-1p33 | −0.429 | 0.338 | L | |
| RP4-737A23 | 1p31.2-1p32.1 | −0.556 | 0.386 | L | |
| RP11-63G10 | 1p31.3-1p31.3; 1p32.1-1p32.3 | −0.401 | 0.504 | L | |
| RP11-89O16 | 1p31-1p31 | −0.373 | 0.486 | L | |
| RP4-662P1 | 1p31.3-1p32.3 | −0.525 | 0.552 | L | |
| RP4-534K7 | 1p31.2-1p32.3 | −0.482 | 0.394 | L | |
| RP11-75N16 | 1p32-1p32 | −0.591 | 0.383 | L | |
| RP11-26A10 | 1p31.2-1p32.3 | −0.447 | 0.576 | L | |
| RP11-89K2 | 1p31.3-1p31.3 | −0.585 | 0.255 | L | |
| RP6-65F20 | 1p32.2-1p34.1(SC) | −0.488 | 0.616 | L | |
| RP4-685B19 | 1p31.2-1p32.2 | −0.669 | 0.309 | L | |
| RP11-79O23 | 1p31.1-1p31.1 | −0.434 | 0.367 | L | |
| RP11-89D5 | 1p31.3-1p31.3 | −0.267 | 0.59 | L | |
| RP4-612J11 | 1p31.2-1p32.1 | −0.803 | 0.582 | L | |
| RP11-492C3 | 1p31.1-1p31.3 | −0.422 | 0.239 | L | |
| RP5-1153M13 | 1p31.1-1p31.3 | −0.486 | 0.672 | L | |
| RP11-80G24 | 1p31.1-1p31.2 | −0.273 | 0.367 | L | |
| RP5-831O21 | 1p31.1-1p31.3 | −0.614 | 0.484 | L | |
| RP4-572F19 | 1p22.3-1p31.3 | −0.491 | 0.541 | L | |
| RP5-989D17 | 1p22.3-1p31.1 | −0.547 | 0.406 | L | |
| RP11-79I13 | 1p22-1p31 | −0.465 | 0.522 | L | |
| RP5-896C23 | 1p22.3-1p31.2 | −0.214 | 0.232 | L | |
| RP11-78E18 | 1p31.1-1p31.1 | −0.211 | 0.385 | L | |
| RP5-1027O11 | 1p22.1-1p22.3 | −0.797 | 0.641 | L | |
| RP5-905H16 | 1p22.1-1p22.3 | −0.568 | 0.513 | L | |
| RP5-1007M22 | 1p22.1-1p22.3 | −0.698 | 0.398 | L | |
| RP5-871E2 | 1p22.2-1p31.1 | −0.313 | 0.504 | L | |
| RP11-99A8 | 1p22.1-1p22.3 | −0.322 | 0.518 | L | |
| RP4-713B5 | 1p21.2-1p22.2 | −0.367 | 0.206 | L | |
| RP11-48A6 | 1p21.3-1p22.3 | −0.272 | 0.221 | L | |
| RP11-122C9 | 1p21.2-1p22.1 | −0.238 | 0.344 | L | |
| RP4-672J20 | 1p21.3-1p22.2 | −0.61 | 0.668 | L | |
| RP11-90N15 | 1p21.3-1p22.1 | −0.6 | 0.305 | L | |
| RP11-411H5 | 1p13.3-1p21.3 | −0.581 | 0.484 | L | |
| RP11-335D10 | 1p21.3-1p22.3(SC) | −0.231 | 0.446 | L | |
| RP11-79H19 | 1p21-1p21 | −0.368 | 0.568 | L | |
| RP11-259N12 | 1p13.3-1p21.3 | −0.496 | 0.797 | L | |
| RP4-669H10 | 1p21.1-1p21.1 | −0.305 | 0.401 | L | |
| RP5-1077K16 | 1p13.3-1p21.1 | −0.393 | 0.312 | L | |
| RP11-96F24 | 1p12-1p13; 1p13.3d-1p13.3d | −0.574 | 0.385 | L | |
| RP11-180N18 | 1p13.2-1p21.1 | −0.512 | 0.398 | L | |
| RP5-1125M8 | 1p13.1-1p13.3 | −0.37 | 0.364 | L | |
| RP4-773A18 | 1p13.2-1p21.1 | −0.257 | 0.498 | L | |
| RP4-787H6 | 1p12-1p13.2 | −0.485 | 0.455 | L | |
| RP11-90J3 | 1p13.1-1p13.1 | −0.457 | 0.505 | L | |
| RP11-88D6 | 1p12-1p12 | −0.231 | 0.332 | L | |
| RP11-433J22 | 1q12-1q21.3 | 0.337 | −0.306 | G | Complete gain of 1q arm |
| RP11-45817 | 1q21.1-1q21.3 | 0.269 | −0.263 | G | except for RP5-1016N21 (next |
| RP4-790G17 | 1q21.1-1q21.3 | 0.273 | −0.251 | G | page) which shows loss. |
| RP11-81P11 | 1q21.2-1q21.2 | 0.552 | −0.283 | G | |
| RP1-148L21 | 1q21.2-1q22 | 0.433 | −0.508 | G | |
| RP11-98G7 | 1q21.2-1q22 | 0.252 | −0.354 | G | |
| RP11-77I10 | 1q21.3-1q23.1 | 0.219 | −0.337 | G | |
| RP11-79M15 | 1q22-1q22 | 0.484 | −0.37 | G | |
| RP11-260G23 | 1q21-1q22; 1q23.3c-1q23.3d | 0.319 | −0.295 | G | |
| RP11-90A11 | 1q22-1q22 | 0.286 | −0.244 | G | |
| RP11-80B20 | 1q22-1q22 | 0.302 | −0.219 | G | |
| RP11-354K16 | 1q21.3-1q23.1 | 0.633 | −0.23 | G | |
| RP1-9E21 | 1q24-1q25 | 0.361 | −0.489 | G | |
| RP11-89P2 | 1q24-1q24 | 0.495 | −0.38 | G | |
| RP11-81H19 | 1q24-1q24 | 0.355 | −0.302 | G | |
| RP11-469I6 | 1q23.3-1q25.1 | 0.323 | −0.499 | G | |
| RP1-105D12 | 1q24-1q25 | 0.442 | −0.534 | G | |
| RP3-395P12 | 1q24-1q25 | 0.336 | −0.214 | G | |
| RP11-415M14 | 1q24.1-1q24.3 | 0.283 | −0.324 | G | |
| RP11-91K17 | 1q24-1q24 | 0.47 | −0.403 | G | |
| RP11-90C19 | 1q24-1q24 | 0.245 | −0.429 | G | |
| RP4-593C16 | 1q24.1-1q25.2 | 0.207 | −0.509 | G | |
| RP11-317P15 | 1q25.2-1q31.2; 1q25-1q31 | 0.424 | −0.254 | G | |
| RP11-63O2 | 1q25.2-1q31.1 | 0.221 | −0.312 | G | |
| RP1-53A19 | 1q25.1-1q31.1 | 0.493 | −0.797 | G | |
| RP11-79I7 | 1q25-1q31 | 0.226 | −0.272 | G | |
| RP11-71C11 | 1q25-1q31 | 0.488 | −0.37 | G | |
| RP3-419C19 | 1q31-1q31 | 0.643 | −0.775 | G | |
| RP11-101E13 | 1q31.2-1q31.3; 1q31-1q31 | 0.233 | −0.257 | G | |
| RP11-358A9 | 1q31.3-1q32.1 | 0.351 | −0.215 | G | |
| RP11-88D12 | 1q31.3-1q31.3 | 0.436 | −0.402 | G | |
| RP11-91G12 | 1q31.3-1q31.3 | 0.423 | −0.343 | G | |
| RP11-80N24 | 1q31.3-1q32.1 | 0.493 | −0.221 | G | |
| RP11-543G21 | 1q31.3-1q32.1 | 0.28 | −0.226 | G | |
| RP11-243M13 | 1q32.1-1q32.1 | 0.37 | −0.338 | G | |
| RP11-79M12 | 1q32-1q32 | 0.265 | −0.47 | G | |
| RP11-79H5 | 1q41-1q41 | 0.241 | −0.236 | G | |
| RP11-260A10 | 1q41 | 0.484 | −0.366 | G | |
| RP11-66M7 | 1q41-1q41 | 0.316 | −0.287 | G | |
| RP11-135J2 | 1q41-1q41 | 0.326 | −0.36 | G | |
| RP11-553F10 | 1q41-1q42.1 | 0.618 | −0.416 | G | |
| RP11-543E8 | 1q42.2-1q43 | 0.308 | −0.212 | G | |
| RP5-1016N21 | 1q42.13-1q43 | −0.361 | 0.492 | L | |
| RP4-781K5 | 1q42.1-1q43 | 0.358 | −0.312 | G | |
| RP4-670F13 | 1q42.2-1q43 | 0.238 | −0.287 | G | |
| RP11-80P14 | 1q43-1q43 | 0.371 | −0.228 | G | |
| RP11-136B18 | 1q42.2-1q43 | 0.264 | −0.399 | G | |
| RP11-90L13 | 1q43-1q43 | 0.511 | −0.457 | G | |
| RP11-81J5 | 1q43-1q43 | 0.284 | −0.377 | G | |
| RP1-241M7 | 1q43-1q44 | 1.128 | −0.488 | G | |
| RP11-88H4 | 1q44-1q44 | 0.469 | −0.276 | G | |
| RP11-407H12 | 1q44 | 0.255 | −0.396 | G | |
| RP11-438F14 | 1q44 | 0.484 | −0.474 | G | |
| CTB-167K11 | 1qter | 0.535 | −0.279 | G | |
| RP11-140B20 | 2q14.3-2q21 | 0.374 | −0.409 | G | Single feature change |
| RP11-79E3 | 4p15.1-4p15.1 | −0.292 | 0.349 | L | Single feature change |
| RP11-17P8 | 4q22-4q22 | 0.348 | −0.334 | G | Single feature change |
| RP11-177L7 | 4q32-4q32 | 0.22 | −0.433 | G | Single feature change |
| AC140172.3 | 5p14.3-5p14.3 | 0.284 | −0.225 | G | Single feature change |
| RP11-626B22 | 5q35.3 | −0.249 | 0.258 | L | Single feature change |
| RP11-80L16 | 6q12-6q12 | −0.219 | 0.525 | L | Single feature change |
| RP4-676J13 | 6q14-6q14 | −0.234 | 0.251 | L | Single feature change |
| AL137072.8 | 9q31.1 | 0.269 | −0.21 | G | Single feature change |
| RP11-145E17 | 9q34.2 | 0.235 | −0.253 | G | Single feature change |
| RP11-164C1 | 10p15.3 | 0.227 | −0.273 | G | Single feature change |
| RP11-89C21 | 10p13-10p14 | −0.53 | 0.629 | L | Significant change, possible |
| | | | | | loss. |
| RP11-79A2 | 10q21-10q21 | −0.344 | 0.232 | L | Single feature change |
| RP11-80N10 | 13q31-13q31 | −0.333 | 0.234 | L | Single feature change |
| RP11-74A12 | 13q31.2-13q32.2 | 0.222 | −0.206 | G | Single feature change |
| RP11-98N22 | 14q11.2-14q11.2 | −0.342 | 0.349 | L | Trend shows complete loss |
| RP11-89F2 | 14q11.2-14q11.2 | −0.212 | 0.257 | L | of chromosome. |
| RP11-71E6 | 14q11.2-14q11.2 | −0.38 | 0.238 | L | |
| RP11-566I2 | 14q11.2c-14q11.2c | −0.515 | 0.365 | L | |
| RP11-65O3 | 14q11.2-14q11.2 | −0.448 | 0.528 | L | |
| RP11-81F9 | 14q11.2-14q11.2 | −0.51 | 0.319 | L | |
| RP11-89K22 | 14q12-14q12 | −0.489 | 0.505 | L | |
| RP11-125A5 | 14q12-14q12 | −0.438 | 0.265 | L | |
| RP11-54H22 | 14q13-14q13 | −0.441 | 0.416 | L | |
| RP11-557O15 | 14q12-14q13.1 | −0.514 | 0.559 | L | |
| RP11-91H1 | 14q21.1-14q21.1 | −0.821 | 0.716 | L | |
| RP11-305B23 | 14q21.1b-14q21.1b | −0.502 | 0.436 | L | |
| RP11-88N14 | 14q21-14q21 | −0.307 | 0.42 | L | |
| RP11-89H24 | 14q21-14q21 | −0.219 | 0.433 | L | |
| RP11-453F20 | 14q21.2-14q21.3 | −0.284 | 0.636 | L | |
| RP11-91J1 | 14q21-14q21 | −0.292 | 0.249 | L | |
| RP11-368A1 | 14q22.1c-14q22.1c | −0.368 | 0.582 | L | |
| RP11-90K14 | 14q21-14q22 | −0.463 | 0.593 | L | |
| RP11-12P7 | 14q22-14q22 | −0.331 | 0.302 | L | |
| RP11-89J8 | 14q32-14q32 | −0.374 | 0.538 | L | |
| RP11-172G1 | 14q22-14q22 | −0.385 | 0.387 | L | |
| RP11-571J17 | 14q23.1a-14q23.1a | −0.588 | 0.5 | L | |
| RP11-2L22 | 14q23-14q23 | −0.328 | 0.398 | L | |
| RP11-79M1 | 14q23-14q23 | −0.451 | 0.373 | L | |
| RP11-471N20 | 14q23.1b-14q23.1c | −0.234 | 0.376 | L | |
| RP11-79I3 | 14q22-14q23 | −0.435 | 0.45 | L | |
| RP11-445J13 | 14q23.1-14q23.2 | −0.363 | 0.366 | L | |
| RP11-63G22 | 14q23-14q23 | −0.208 | 0.419 | L | |
| RP11-156E22 | 14q23-14q23 | −0.321 | 0.242 | L | |
| RP11-79B13 | 14q24-14q24 | −0.267 | 0.492 | L | |
| AL160191.3 | 14q24.2 | −0.382 | 0.637 | L | |
| RP11-325N20 | 14q24.2a-14q24.2b | −0.366 | 0.584 | L | |
| RP11-382O4 | 14q24.3a-14q24.3a | −0.503 | 0.324 | L | |
| CTD-2317F5 | 14q24.3-14q24.3 | −0.46 | 0.501 | L | |
| RP11-81O20 | 14q24.3-14q24.3 | −0.51 | 0.453 | L | |
| RP11-63D17 | 14q24.3-14q31 | −0.375 | 0.352 | L | |
| CTD-3014H8 | 14q24.3-14q24.3 | −0.382 | 0.519 | L | |
| RP11-232C2 | 14q24.3-14q24.3 | −0.601 | 0.38 | L | |
| RP11-80L10 | 14q31.1-14q31.1 | −0.291 | 0.375 | L | |
| RP11-114N19 | 14q24-14q31 | −0.303 | 0.507 | L | |
| AL133279.7 | 14q31.3 | −0.409 | 0.567 | L | |
| RP11-79J20 | 14q32.1-14q32.1 | −0.497 | 0.408 | L | |
| RP11-99C24 | 14q32.1-14q32.1 | −0.606 | 0.309 | L | |
| RP11-374H13 | 14q32.12-14q32.12 | −0.568 | 0.304 | L | |
| RP11-160P21 | 14q32.2-14q32.2 | −0.285 | 0.47 | L | |
| RP11-88L4 | 14q32.2-14q32.2 | −0.864 | 0.437 | L | |
| RP11-431B1 | 14q32.2b-14q32.2b | −0.624 | 0.721 | L | |
| RP11-90G22 | 14q32.2-14q32.2 | −0.524 | 0.497 | L | |
| RP11-365N19 | 14q32.32 | −0.218 | 0.209 | L | |
| RP11-73M18 | 14q32.33 | −0.261 | 0.219 | L | |
| RP5-820M16 | 14q32.33 | −0.41 | 0.545 | L | |
| AC009054.8 | 16q23.1 | 0.202 | −0.227 | G | Single feature change |
| RP1-191P24 | 16q24.3 | 0.475 | −0.203 | G | Single feature change |
| RP11-89F21 | 17p11.2-17p12 | −0.382 | 0.208 | L | |
| RP11-79O18 | 17q21-17q21 | 0.303 | −0.416 | G | Significant change, possible |
| | | | | | gain. |
| CTB-74G18 | 18p11.32 | −0.418 | 0.318 | L | Trend shows complete loss |
| RP11-55N14 | 18p11.32a-18p11.32a | −0.564 | 0.394 | L | of chromosome 18. |
| RP11-80L18 | 18p11.31-18p11.31 | −0.465 | 0.463 | L | |
| RP11-105C15 | 18p11.31c-18p11.31c | −0.662 | 0.466 | L | |
| RP11-91I8 | 18p11.2-18p11.3 | −0.562 | 0.521 | L | |
| RP11-102O20 | 18p11.22c-18p11.22c | −0.321 | 0.462 | L | |
| AP001077.5 | 18p11.21 | −0.273 | 0.497 | L | |
| RP11-151D11 | 18p11.21e-18p11.21e | −0.507 | 0.46 | L | |
| RP11-411B10 | 18p11.2-18p11.2 | −0.459 | 0.233 | L | |
| RP11-79F3 | 18q11.2-18q11.2 | −0.447 | 0.551 | L | |
| RP11-676D16 | 18q11.2-18q11.2 | −0.533 | 0.474 | L | |
| RP11-79G13 | 18q12-18q12 | −0.284 | 0.43 | L | |
| AC021224.7 | 18q12.1g-18q12.1g | −0.314 | 0.654 | L | |
| RP11-63N12 | 18q12.1g-18q12.1g | −0.313 | 0.493 | L | |
| RP11-90B5 | 18q12.2b-18q12.2c; | −0.315 | 0.655 | L | |
| | 18q12-18q12 | | | | |
| RP11-104N11 | 18q12.2d-18q12.2d | −0.557 | 0.828 | L | |
| RP11-89M10 | 18q12.3-18q12.3 | −0.428 | 0.499 | L | |
| RP11-20A13 | 18q12.3-18q12.3 | −0.303 | 0.41 | L | |
| RP11-91K12 | 18q12.3-18q21.1 | −0.764 | 0.504 | L | |
| RP11-80P2 | 18q21.1-18q21.1 | −0.282 | 0.304 | L | |
| RP11-160B24 | 18q21.2d-18q21.2d | −0.383 | 0.526 | L | |
| RP11-153B11 | 18q21.31a-18q21.31b | −0.536 | 0.431 | L | |
| RP11-79L5 | 18q21.2-18q21.2 | −0.474 | 0.4 | L | |
| RP11-4G8 | 18q21.31-18q21.32 | −0.482 | 0.27 | L | |
| RP11-75O12 | 18q21.33b-18q21.33b | −0.452 | 0.482 | L | |
| RP11-90B3 | 18q21.3-18q22 | −0.407 | 0.548 | L | |
| RP11-89I22 | 18q22-18q22 | −0.316 | 0.508 | L | |
| RP11-88B2 | 18q22-18q22 | −0.455 | 0.42 | L | |
| RP11-105L16 | 18q22.1e-18q22.1f | −0.685 | 0.593 | L | |
| RP11-79A24 | 18q22-18q22 | −0.31 | 0.588 | L | |
| RP11-90A7 | 18q22-18q22 | −0.432 | 0.631 | L | |
| RP11-49H23 | 18q22.2-18q22.2 | −0.299 | 0.715 | L | |
| RP11-57F7 | 18q22.3a-18q22.3b | −0.487 | 0.494 | L | |
| RP11-90L15 | 18q23-18q23 | −0.259 | 0.41 | L | |
| AC096709.19 | 18q23 | −0.301 | 0.432 | L | |
| RP11-90L3 | 18q23-18q23 | −0.473 | 0.389 | L | |
| RP11-91C19 | 18q23-18q23 | −0.728 | 0.395 | L | |
| RP11-89N1 | 18q23-18q23 | −0.304 | 0.571 | L | |
| RP5-1103G7 | 20p12.2-20p13 | 0.238 | −0.358 | G | |
| RP11-91O6 | 22q11.2-22q11.2 | −0.546 | 0.467 | L | Single feature change |
| RP11-81B3 | 22q11.2-22q11.2 | −0.55 | 0.39 | L | Complete loss of chromosome 22 |
| RP11-186O8 | 22q11.1-22q11.22 | −0.411 | 0.391 | L | except for RP5-1119A7 at 22q12.2-22q12.3. |
| RP11-76E8 | 22q12.1-22q12.1 | −0.903 | 0.483 | L | |
| RP11-89A2 | 22q12.1-22q12.1 | −0.261 | 0.362 | L | |
| RP11-91K24 | 22q12.1-22q12.1 | −0.394 | 0.67 | L | |
| RP11-79G21 | 22q12-22q12 | −0.534 | 0.553 | L | |
| RP11-79G6 | 22q12-22q12 | −0.453 | 0.514 | L | |
| RP11-247I13 | 22q12-22q12 | −0.412 | 0.301 | L | |
| Z73979.1 | 22q12.3 | −0.486 | 0.344 | L | |
| RP1-215F16 | 22q12.1-22q12.3 | −0.766 | 0.318 | L | |
| RP3-327J16 | 22q12.3-22q13.2 | −0.733 | 0.403 | L | |
| RP3-370M22 | 22q13.1-22q13.2 | −0.59 | 0.256 | L | |
| RP5-821D11 | 22q12.3-22q13.1 | −0.43 | 0.245 | L | |
| RP1-185D5 | 22q13.1-22q13.31 | −0.252 | 0.424 | L | |
| RP1-32I10 | 22q13.2-22q13.31 | −1.086 | 0.428 | L | |
| RP11-140I15 | 22q13.2-22q13.33 | −0.623 | 0.461 | L | |
| RP5-1163J1 | 22q13.2-22q13.33 | −0.559 | 0.266 | L | |
| RP1-111J24 | 22q12.2-22q12.3 | −0.631 | 0.273 | L | |
| RP11-262A13 | 22q13.2-22q13.33 | −0.383 | 0.318 | L | |
| U62317.2 | 22q13.33 | −0.318 | 0.245 | L | |

Many single changes seen are probably due to noise in the specimen, control or array. Those single feature changes that achieve greater significance (out to or near the first dotted lines) are called out as suspicious. Those single feature changes near or on the significance lines (blue) are regarded as probable noise
Sex Mismatch Controls.
X: Expected gain of X chromosome.
Y: Expected loss of entire Y chromosome

This data was then imported into DecisionSite 8.1.1 software (Spotfire, Sommerville, Mass.) for analysis based on the Taxol EDR status. Only regions that were changed in a statistically-significant manner were used for further analyses. Genetic loci to be studied further were selected from all the data where at least four of the sixteen Taxol EDR tumors possessed the change and less than one of the six Taxol LDR tumors possessed the change. These differences are in the tables in FIGS. 12 and 13.

The table below is a listing of the BACs and the genetic locations that met the criteria for further analyses of amplified regions following analyses of the data in Spotfire. The criteria were that the amplification had to be present in at least four Taxol EDR tumors and one or less of the Taxol LDR tumors.

| Clone | Cyto Locn | EDR GAIN | LDR GAIN | EDR LOSS | LDR LOSS | EDR No Change | LDR No Change |
|---|---|---|---|---|---|---|---|
| RP4-790G17 | 1q21.1-1q21.3 | 10 | 1 | 0 | 0 | 4 | 6 |
| RP4-703E10 | 1p36.32-1p36.33 | 9 | 1 | 1 | 0 | 4 | 6 |
| RP11-81P11 | 1q21.2-1q21.2 | 8 | 1 | 0 | 0 | 6 | 6 |
| RP11-98G7 | 1q21.2-1q22 | 7 | 0 | 2 | 0 | 5 | 7 |
| RP11-407H12 | 1q44 | 7 | 0 | 1 | 0 | 6 | 7 |
| RP11-317P15 | 1q25.2-1q31.2; 1q25-1 | 6 | 0 | 0 | 0 | 8 | 7 |
| RP4-781K5 | 1q42.1-1q43 | 6 | 0 | 0 | 0 | 8 | 7 |
| RP11-438F14 | 1q44 | 6 | 0 | 1 | 0 | 7 | 7 |
| RP11-90M17 | 6p22.3-6p23 | 6 | 0 | 1 | 0 | 7 | 7 |
| RP1-224B21 | 6p21.32-6p22.2 | 6 | 0 | 0 | 0 | 8 | 7 |
| RP1-52M20 | 6p22.1-6p22.3 | 5 | 0 | 0 | 1 | 9 | 6 |
| AC120042.3 | 8q12.3 | 5 | 0 | 0 | 0 | 9 | 7 |
| AC009879.8 | 8q13.1 | 5 | 0 | 0 | 0 | 9 | 7 |
| RP11-64J22 | 12p12-12p12 | 5 | 0 | 0 | 0 | 9 | 7 |
| RP11-452O22 | 1q21.1-1q22 | 5 | 1 | 0 | 0 | 9 | 6 |
| RP1-53A19 | 1q25.1-1q31.1 | 5 | 1 | 0 | 0 | 9 | 6 |
| RP11-492C3 | 1p31.1-1p31.3 | 4 | 0 | 1 | 0 | 9 | 7 |
| RP4-672J20 | 1p21.3-1p22.2 | 4 | 0 | 3 | 1 | 7 | 6 |
| RP5-1090A23 | 1q41-1q42.2 | 4 | 0 | 0 | 0 | 10 | 7 |
| RP11-90L13 | 1q43-1q43 | 4 | 0 | 0 | 0 | 10 | 7 |
| RP11-91A9 | 2q24.3-2q31 | 4 | 0 | 0 | 0 | 10 | 7 |
| RP11-79C17 | 2q31-2q31 | 4 | 0 | 0 | 0 | 10 | 7 |
| RP11-270G18 | 2q32-2q32 | 4 | 0 | 0 | 0 | 10 | 7 |
| RP11-49H14 | 4q21-4q21 | 4 | 0 | 3 | 0 | 7 | 7 |
| RP1-84C11 | 5pter | 4 | 0 | 0 | 0 | 10 | 7 |
| RP1-136B1 | 6p24.1-6p25.3 | 4 | 0 | 1 | 0 | 9 | 7 |
| RP11-304M10 | 6p24.1-6p25.3 | 4 | 0 | 2 | 0 | 8 | 7 |
| RP3-365E2 | 6p22.3-6p24.1 | 4 | 0 | 1 | 0 | 9 | 7 |
| RP1-298J15 | 6p22.3-6p23 | 4 | 0 | 0 | 0 | 10 | 7 |
| RP1-130G2 | 6p22.2-6p22.3 | 4 | 0 | 2 | 0 | 8 | 7 |
| RP11-91H17 | 6p22-6p22 | 4 | 0 | 1 | 0 | 9 | 7 |
| RP3-369A17 | 6p22.1-6p22.3 | 4 | 0 | 0 | 0 | 10 | 7 |
| RP5-874C20 | 6p22.1-6p22.3 | 4 | 0 | 0 | 0 | 10 | 7 |
| AC046176.13 | 8q12.1 | 4 | 0 | 0 | 0 | 10 | 7 |
| RP11-120-N14 | 8q13.3 | 4 | 0 | 0 | 0 | 10 | 7 |
| RP11-80F23 | 14q32.2-14q32.2 | 4 | 0 | 0 | 0 | 10 | 7 |
| RP5-1005F21 | 20q13.33 | 4 | 0 | 2 | 0 | 8 | 7 |
| RP11-137P24 | 1q21.1-1q21.3 | 4 | 1 | 0 | 0 | 10 | 6 |
| RP11-767C6 | 8q11.23d-8q11.23d | 4 | 1 | 0 | 0 | 10 | 6 |
| RP11-172D2 | 8q12-8q12 | 4 | 1 | 0 | 0 | 10 | 6 |
| RP11-89I14 | 8q21.1-8q21.1 | 4 | 1 | 0 | 0 | 10 | 6 |
| RP11-449D3 | 8q24.3-8q24.3 | 4 | 1 | 0 | 0 | 10 | 6 |
| RP11-489O18 | 8q24.23b-8q24.23b | 4 | 1 | 0 | 0 | 10 | 6 |
| RP11-543P15 | 12p13.32b-12p13.32b | 4 | 1 | 0 | 0 | 10 | 6 |
| RP11-122A8 | 13q32-13q32 | 4 | 1 | 0 | 0 | 10 | 6 |

The table below is a listing of the BACs and the genetic locations that met the criteria for further analyses of deleted regions following analyses of the data in Spotfire. The criteria were that the deletion had to be present in at least four Taxol EDR tumors and one or less of the Taxol LDR tumors.

| Clone | Cyto Locn | EDR GAIN | LDR GAIN | EDR LOSS | LDR LOSS | EDR No Change | LDR No Change |
|---|---|---|---|---|---|---|---|
| RP11-73G16 | 4q31.1-4q31.1; 4q31.3 | 0 | 0 | 9 | 1 | 5 | 6 |
| RP11-89O4 | 8p22-8p22 | 0 | 0 | 9 | 1 | 5 | 6 |
| RP11-90O9 | 2q12-2q12 | 0 | 0 | 8 | 0 | 6 | 7 |
| RP11-1K11 | 8p23.2 | 0 | 0 | 8 | 1 | 6 | 6 |
| RP11-89I12 | 8p23.2-8p23.2 | 0 | 0 | 8 | 1 | 6 | 6 |
| RP11-90O17 | 8p22-8p22 | 1 | 0 | 7 | 1 | 6 | 6 |
| RP11-23H1 | 8p22c-8p22c | 0 | 0 | 7 | 1 | 7 | 6 |
| RP11-89M8 | 8p21-8p21 | 0 | 0 | 7 | 1 | 7 | 6 |
| RP1-185D5 | 22q13.1-22q13.31 | 0 | 0 | 7 | 1 | 7 | 6 |
| RP11-551B22 | 5q23.1-5q23.1 | 0 | 1 | 7 | 1 | 7 | 5 |
| RP11-349I11 | 16p13.3-16p13.3 | 1 | 0 | 6 | 0 | 7 | 7 |
| RP11-81H11 | 4p15.1-4p15.1 | 0 | 0 | 6 | 0 | 8 | 7 |
| RP11-108H14 | 4p13-4p14 | 0 | 0 | 6 | 0 | 8 | 7 |
| RP11-89B16 | 4q12b-4q12b; 4q12-4q | 0 | 0 | 6 | 0 | 8 | 7 |
| CTB-77L23 | 8p23.3 | 0 | 0 | 6 | 0 | 8 | 7 |
| AC126333.7 | 8p23.3 | 0 | 0 | 6 | 0 | 8 | 7 |
| RP11-780-O22 | 8p23.1 | 0 | 0 | 6 | 0 | 8 | 7 |
| AC010656.7 | 8p22 | 0 | 0 | 6 | 0 | 8 | 7 |
| RP11-77C9 | 11q25a-11q25a | 0 | 0 | 6 | 0 | 8 | 7 |
| RP11-90L3 | 18q23-18q23 | 0 | 0 | 6 | 0 | 8 | 7 |
| Z73979.1 | 22q12.3 | 0 | 0 | 6 | 0 | 8 | 7 |
| RP11-79E11 | 8p21-8p22 | 1 | 0 | 6 | 1 | 7 | 6 |
| RP11-70L1 | 8p21.2-8p21.2 | 1 | 0 | 6 | 1 | 7 | 6 |
| RP11-647P12 | 4q27b-4q27b | 0 | 0 | 6 | 1 | 8 | 6 |
| RP11-45M12 | 8p23-8p23 | 0 | 0 | 6 | 1 | 8 | 6 |
| RP11-76B12 | 8p21.2-8p21.2 | 0 | 0 | 6 | 1 | 8 | 6 |
| RP11-173D10 | 8p12-8p12 | 0 | 0 | 6 | 1 | 8 | 6 |
| U62317.2 | 22q13.33 | 0 | 0 | 6 | 1 | 8 | 6 |
| RP11-22M5 | 22q11.2-22q11.2 | 1 | 0 | 5 | 0 | 8 | 7 |
| RP11-91O13 | 15q23-15q23 | 0 | 0 | 5 | 0 | 9 | 7 |
| RP11-91O6 | 22q11.2-22q11.2 | 0 | 0 | 5 | 0 | 9 | 7 |
| RP11-252K12 | 8p23.1-8p23.1 | 1 | 0 | 5 | 1 | 8 | 6 |
| RP11-237M13 | 8p11.2-8p12 | 1 | 0 | 5 | 1 | 8 | 6 |
| RP11-189B4 | 13q14.11-13q14.3 | 1 | 0 | 5 | 1 | 8 | 6 |
| RP11-125A5 | 14q12-14q12 | 1 | 0 | 5 | 1 | 8 | 6 |
| RP11-558F16 | 15q25.1a-15q25.1b | 1 | 0 | 5 | 1 | 8 | 6 |
| RP3-355C18 | 22q13.3-22q13.3 | 1 | 0 | 5 | 1 | 8 | 6 |
| RP1-140C12 | 6q26-6q27; 6q27-6q27 | 0 | 0 | 5 | 1 | 9 | 6 |
| RP11-79I19 | 8p23-8p23 | 0 | 0 | 5 | 1 | 9 | 6 |
| RP11-90I3 | 8p22-8p22 | 0 | 0 | 5 | 1 | 9 | 6 |
| RP11-139G9 | 8p12-8p12 | 0 | 0 | 5 | 1 | 9 | 6 |
| RP11-89D12 | 22q12-22q12 | 0 | 1 | 5 | 1 | 9 | 5 |
| RP4-534K7 | 1p31.2-1p32.3 | 3 | 0 | 4 | 0 | 7 | 7 |
| RP11-100C24 | 13q14.3-13q21.2; 13q | 3 | 2 | 4 | 0 | 7 | 5 |
| CTD-2004C12 | 5q23-5q31 | 1 | 0 | 4 | 0 | 9 | 7 |
| RP11-429-B7 | 8p23.1 | 1 | 0 | 4 | 0 | 9 | 7 |
| RP11-375-N15 | 8p23.1 | 1 | 0 | 4 | 0 | 9 | 7 |
| RP11-89D3 | 16p13.2-16p13.2; 16p | 1 | 0 | 4 | 0 | 9 | 7 |
| RP11-141E3 | 16p12-16p12 | 1 | 0 | 4 | 0 | 9 | 7 |
| RP11-146J7 | 16p11.2-16p11.2 | 1 | 0 | 4 | 0 | 9 | 7 |
| RP11-81B3 | 22q11.2-22q11.2 | 1 | 0 | 4 | 0 | 9 | 7 |
| RP11-186O8 | 22q11.1-22q11.22 | 1 | 0 | 4 | 0 | 9 | 7 |
| RP11-80L16 | 6q12-6q12 | 0 | 0 | 4 | 0 | 10 | 7 |
| RP11-89N1 | 18q23-18q23 | 0 | 0 | 4 | 0 | 10 | 7 |
| RP11-17I20 | 19q13.3-19q13.3 | 0 | 0 | 4 | 0 | 10 | 7 |
| RP5-930L11 | 22q11.21-22q11.23 | 0 | 0 | 4 | 0 | 10 | 7 |
| RP4-695O20 | 22q13.1-22q13.33(SO | 0 | 0 | 4 | 0 | 10 | 7 |
| RP11-24M13 | 16p13.2-16p13.2 | 1 | 0 | 4 | 1 | 9 | 6 |
| RP1-57H24 | 6q27 | 0 | 0 | 4 | 1 | 10 | 6 |
| RP11-164A10 | 11q24-11q24 | 0 | 0 | 4 | 1 | 10 | 6 |
| RP11-79G6 | 22q12-22q12 | 0 | 0 | 4 | 1 | 10 | 6 |
| RP11-247I13 | 22q12-22q12 | 0 | 0 | 4 | 1 | 10 | 6 |

These results established that tumors that were EDR to paclitaxel and docletaxel showed resistance-associated amplification of human chromosome 1q, 2q, 6p, 8q, or 14q or deletion of human chromosome 20q, 2q, 4p, 4q, 5q, 6q, 8p, 11, 13q, 14q, 15q, 16p, or 18q in breast, ovarian and non-small cell lung cancer. These results further confirmed that tumors showing the observed amplification patterns for genes and genetic loci on human chromosome 1q, 2q, 6p, 8q, or 14q or deletion of human chromosome 20q, 2q, 4p, 4q, 5q, 6q, 8p, 11, 13q, 14q, 15q, 16p, or 18q were poor candidates for chemotherapeutic intervention with paclitaxel or docletaxel. These results provided a more efficient and cost-effective alternative to screening tumor samples using the EDR assay.

It should be understood that the foregoing disclosure emphasizes certain specific embodiments of the invention and that all modifications or alternatives equivalent thereto are within the spirit and scope of the invention as set forth in the appended claims.

We claim:

1. A method for identifying a human tumor that is resistant to a chemotherapeutic drug, comprising the step of assaying one or a plurality of genes or genetic loci from a chromosomal region in the tumor for amplification or overexpression of said genes or genetic loci, wherein the tumor is determined to be resistant to the chemotherapeutic drug if one or the plurality of genes or genetic loci is amplified or overexpressed in the tumor.

2. The method of claim 1 wherein the tumor is a breast cancer tumor, ovarian cancer tumor, or non-small cell lung cancer tumor.

3. The method of claim 1 wherein the drug is paclitaxel, docetaxel or epithilones.

4. The method of claim 1 wherein when the chromosomal region is amplified the amplified region is located at chromosome 1q21-1q25, 1q32, 1q41, 1q42, 1q43, 1q44, 2q31, 6p22-25, 8q12, or 14q32, and when the chromosomal region is deleted the deleted region is located at chromosome 20q13, 2q12, 4p12-15, 4q27-31, 5q23, 6q26, 8p11-23, 11q25, 13q14, 14q12, 15q23-25, 16p11-13, or 18q23.

5. The method of claim 1 wherein the genes are H2BFQ, SLC19A2, ZNF281, DAF or ATF3.

6. The method of claim 1, wherein the tumor cells are separated from the tumor sample.

7. The method of claim 1, wherein amplification of one or a plurality of genes or genetic loci from a chromosomal region are detected by gene arrays, fluorescence in situ hybridization (FISH), Southern blot, polymerase chain reaction (PCR), enzyme-linked immunosorbent assay (ELISA) or comparative genomic hybridization (CGH).

8. The method of claim 1, wherein overexpression of one or a plurality of genes or genetic loci are detected by assaying RNA expression.

9. The method of claim 8, wherein RNA overexpression is detected by gene expression arrays, RT-PCR, or Northern blots.

10. The method of claim 1, wherein overexpression of one or a plurality of genes is detected by assaying protein expression.

11. The method of claim 10, wherein protein overexpression is detected by western blot, ELISA, immunohistochemistry or mass spectroscopy.

12. A kit for assaying amplification or overexpression of one or a plurality of genes or genetic loci, comprising at least one probe specific for any said genes or genetic loci.

13. The kit of claim 12 wherein the amplified genes or genetic loci is located at chromosome 1q21-1q25are 1q21-1q25, 1q32, 1q41, 1q42, 1q43, 1q44, 2q31, 6p22-25, 8q12, or 14q32.

14. The kit of claim 13, wherein the genes are H2BFQ, SLC19A2, ZNF281, DAF or ATF3.

15. The kit according to claim 12, further comprising a detectable label for labeling each of said probes.

16. The kit of claim 12, wherein the probes are detectably labeled.

17. One or a plurality of probes for a plurality of genes or genetic loci that are amplified or overexpressed in a tumor that is resistant to a chemotherapeutic drug, wherein each probe specifically binds or hybridizes to a gene or genetic locus located within 1q21-1q25, 1q32, 1q41, 1q42, 1q43, 1q44, 2q31, 6p22-25, 8q12, 14q32, 20q13, 2q12, 4p12-15, 4q27-31, 5q23, 6q26, 8p11-23, 11q25, 13q14, 14q12, 15q23-25, 16p11-13, or 18q23.

18. The probes of claim 17 that specifically bind or hybridize to H2BFQ, SLC19A2, ZNF281, DAF or ATF3.
